(19) United States
(12) Patent Application Publication
(10) Pub. No.: US 2025/0375528 A1
(43) Pub. Date: Dec. 11, 2025

(54) CONJUGATE OF CELL-PENETRATING PEPTIDE AND MELPHALAN, AND PREPARATION CONTAINING CONJUGATE

(71) Applicant: ALEPHOSON BIOPHARMACEUTICALS LIMITED, Shatin (CN)

(72) Inventors: Gang WEI, Shanghai (CN); Kuan JIANG, Shanghai (CN); Benjamin Tak Kwong LEE, Shatin (CN); Yanfeng WANG, Tai Wai (CN)

(21) Appl. No.: 18/580,492

(22) PCT Filed: Jul. 21, 2022
(86) PCT No.: PCT/CN2022/107105

§ 371 Date: Jan. 18, 2024

(30) Foreign Application Priority Data
Jul. 21, 2021 (CN) 202110824706.X

(51) Int. Cl. A61K 47/64; A61K 9/08; A61K 31/198; A61P 27/02

(52) U.S. Cl. CPC A61K 47/64; A61K 9/08; A61K 31/198; A61P 27/02

(57) ABSTRACT

The present invention relates to a conjugate of cell-penetrating peptide and melphalan and a formulation comprising the same. Specifically, the present invention relates to a conjugate formed by covalently linking cell-penetrating peptide derivative to melphalan, a formulation comprising the conjugate, a method for treating diseases with the conjugate, and the application of the formulation in treating diseases.

### TECHNICAL FIELD

The present invention belongs to pharmaceutical field, and relates to a conjugate formed by covalently linking cell-penetrating peptide (Penetratin) derivative to a small molecule drug, a formulation comprising the conjugate, a method for treating diseases with the conjugate, and the application of the formulation in treating diseases.

### BACKGROUND OF THE INVENTION

Retinoblastoma (RB) is the most common intraocular malignancy in children, with a global incidence of about 1/15,000-1/20,000 and about 9,000 new cases every year, causing serious social and family burdens. During the fetal eye development, the allelic mutation of the retinoblastoma gene RB1 in sensitive retinal cells (e.g., photosensitive precursor cells) will cause a benign tumor “retinoma” in the retina. Thereafter, if malignant proliferation (i.e., RB) occurs, “leukocoria” will form and then affect the vision seriously. If not treated in time, further malignant proliferation of the tumor will cause the optic nerve and central system metastasis as well as the extraorbital bone metastasis, etc., and finally cause death in the affected children (if brain metastasis occurs, mortality is almost 100%). Currently, ophthalmectomy or extraocular radiotherapy is clinically mature for the treatment of RB. However, as to the early-stage RB, chemotherapy is the only way to save the eyes, the administration modes including intravitreal injection, periocular injection, arterial infusion, intravenous administration, etc., and the representative drugs including melphalan, vincristine, etoposide, carboplatin, methotrexate, topotecan, etc.

In the current market, more than 90% of the ophthalmic drugs are small molecule drug eye drops. Due to the absorption barriers in the anterior ocular segment, such as tear clearance, corneal epithelial barrier, etc., the bioavailability of traditional eye drops is usually no more than 5%. Moreover, eye drops just have a good controlling effect on anterior ocular segment diseases, while the intraocular injection is still the preferred mode of administration for the posterior ocular segment diseases in clinical. However, the intraocular injection has great influence on the compliance of patients, and the safety and convenience of injection operations are unsatisfactory. Especially, general anesthesia is usually required for the intraocular injections in children. Therefore, there is still a great need for more convenient and more effective modes of administration and drugs in the field.

Cell-penetrating peptides (CPPs) are short peptides with positive charges under physiological pH conditions, and can mediate the entry of the covalently or non-covalently linked molecules or drug delivery systems (e.g., liposomes, nanoparticles, micelles, etc.) into the cells (J. Controlled Release, 2019, 309: 106-124).

CN108976288A disclosed a derivative based on wild-type cell-penetrating peptide, wherein the derivative was covalently coupled with a tracer molecule, e.g., fluorescent probe carboxyl fluorescein (FAM), and the in vivo penetration effect was investigated. However, in CN108976288A, the covalent conjugates of CPPs derivative and small molecule drug were not actually prepared, and the in vivo efficacy of the conjugates was not investigated.

US20190015521 disclosed the use of cell-penetrating peptides (CPPs) for local delivery of therapeutic agents for the treatment of age-related macular degeneration, wherein the therapeutic agents may be mixed with or noncovalently or covalently coupled to CPPs. However, the CPPs disclosed therein are wild-type CPPs, rather than the peptide derivatives with strong membrane-penetrating effect as described in CN108976288A. Moreover, the examples of US20190015521 mainly disclosed the effect of the physical mixture of therapeutic agents and CPPs, and the in vivo efficacy of the covalently coupled product of CPPs and drug has never been confirmed.

Thus, the prior art has never taught that the covalent conjugates of cell-penetrating peptide derivatives and a small molecule drug melphalan, nor did it teach the administration of the conjugates by eye dropping, to say nothing of investigating the in vivo efficacy by administering the eye drops comprising the conjugates, etc.

### CONTENTS OF THE INVENTION

To address the issue of intraocular administration of melphalan, the inventors constructed the polypeptide-small molecule covalent conjugates with the CPPs derivates (see CN108976288A, the content of which is incorporated herein by reference in their entirety) and two model small molecules, including lipophilic small molecule carboxyl fluorescein (FAM) and hydrophilic small molecule water-soluble cyanine dye (sulfo-Cy5), and a small molecule drug melphalan (Mel), which is for intravitreal injection, by means of covalent coupling. The in vitro characterization, cell level evaluation, permeability evaluation in ex vivo tissue, in vivo anti-tumor effect, safety evaluation and the like of the conjugates have also been investigated.

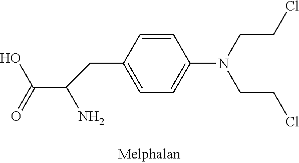 [SMILES: NC(Cc1ccc(N(CCCl)CCCl)cc1)C(=O)O]

Surprisingly, firstly, the solubility of melphalan increases by over 5000 times upon being coupled with the polypeptides, while in the existing melphalan intraocular injections, such organic solvents as propylene glycol and/or ethanol shall be added as the solubilizers, which can easily cause eye irritation and is not conducive to eye administration. When the solubility of melphalan increases, the unnecessary organic solvents can be removed from the formulations, making the formulations more convenient and safer. Secondly, the inventors have found that the non-invasive intraocular delivery of the melphalan covalent conjugates mediated by the polypeptides is feasible. The conjugates can not only significantly improve the bioavailability of the drugs after being dropped into the eyes, particularly improve the absorption of the drugs in the posterior ocular segment, but also achieve the satisfactory in vivo results of both pharmacodynamics and toxicology investigations, the in vivo efficacy thereof being substantially comparable to that of the conventional melphalan intravitreal injection solution. Moreover, the inventors unexpectedly found that the eye drops comprising the covalent conjugates of melphalan and the polypeptides can significantly reduce the brain metastasis proportion of intraocular tumors, the effect of which is even far better than that of conventional melphalan intravitreal injection solution.

### I. Conjugates

In the first aspect, the present invention provides a compound of formula (I), (II) or (III) or a pharmaceutically acceptable salt thereof, which represents a covalent conjugate of cell-penetrating peptide derivative and melphalan,

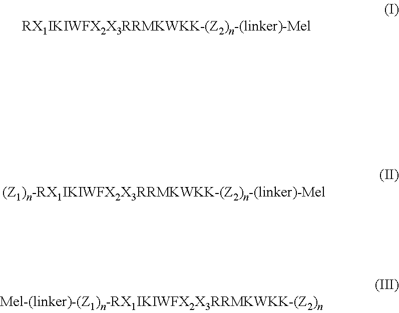

wherein, X₁, X₂ and X₃ represent hydrophobic amino acid, each of which is independently selected from alanine (A), valine (V), leucine (L), isoleucine (I), proline (P), phenylalanine (F), tryptophan (W), methionine (M) and non-naturally occurring amino acids α-aminobutyric acid, α-aminopentanoic acid, α-aminohexanoic acid and α-aminoheptanoic acid;
Z₁ and Z₂ represent naturally or non-naturally occurring amino acids, which is independently selected from 1, 2, 3, 4 or 5 of glycine (G), alanine (A), lysine (K), arginine (R), serine (S), histidine (H), aspartic acid (D), glutamic acid (E), threonine (T), proline (P), cysteine (C), tyrosine (Y), valine (V), methionine (M), isoleucine (I), leucine (L), phenylalanine (F), tryptophan (W), Glutamine (Q), Asparagin (N), and non-naturally occurring hydroxyproline, α-aminobutyric acid, α-aminopentanoic acid, α-aminohexanoic acid and α-aminoheptanoic acid, and the numbers of Z₁ and Z₂ are independent of each other;
n is an integer ranging from 0 to 10, preferably from 0 to 5, for example 0, 1, 2, 3, 4 or 5;
Mel is the melphalan moiety, and
linker represents a covalent bond or a linking group formed between melphalan and the adjacent amino acid, such as amide bond, ester bond, ether bond, disulfide bond, hydrazone, urea, oxime, guanidine, amidine, acetal, imine, alkylene or other linking forms; for example, linker can be selected from the following forms: —O—, —S—, —S—S—, —CH═N—O—, —C₁₋₆ alkylene-, —NH—, —N(R₁)—, —CO—NH—, —CO—N(R₁)—, —C₁₋₆ alkylene-(CO—NH)—, —C₁₋₆ alkylene-(CO—N(R₁))—, —C₁₋₆ alkylene-N(R₁)—, —NH—CO—, —N(R₁)—CO—, —C₁₋₆ alkylene-NH—CO—, —C₁₋₆ alkylene-N(R₁)—CO—, —NH—CO—NH—, —N(R₁)—CO—NH, —NH—CO—N(R₁)—, —N(R₁)—CO—N(R₁), —C(═O)O—, —C₁₋₆ alkylene-C(═O)O—, —C(═O)O—C₁₋₆ alkylene-, —S(═O)NH—, —S(═O)N(R₁)—, —NH—S(═O)—, —N(R₁)S(═O)—, —C₁₋₆ alkylene-S(═O)NH—, —C₁₋₆ alkylene-S(═O)N(R₁)—, —C₁₋₆ alkylene-NH—S(═O)—, —C₁₋₆ alkylene-N(R₁)S(═O)—, —S(═O)₂—NH—, —S(═O)₂—N(R₁)—, —NH—S(═O)₂—, —N(R₁)S(═O)₂—, —C₁₋₆ alkylene-S(═O)₂—NH—, —C₁₋₆ alkylene-S(═O)₂—N(R₁)—, —C₁₋₆ alkylene-NH—S(═O)₂—, —C₁₋₆ alkylene-N(R₁)S(═O)₂—, or C₁₋₆ alkylene substituted with one or more R₁; wherein R₁ is selected from C₁₋₆ alkyl, C₃₋₈ cycloalkyl, C₆₋₁₀ aryl, 5- to 10-membered heteroaryl or 3- to 12-membered heterocycloalkyl, each of which is optionally substituted with one or more groups independently selected from halogen, amino, —NH(C₁₋₆ alkyl), —N(C₁₋₆ alkyl)₂, hydroxyl, C₁₋₆ alkyl, C₂₋₆ alkenyl, C₂₋₆ alkynyl, C₁₋₆ alkoxy, halogen-substituted C₁₋₆ alkyl, halogen-substituted C₁₋₆ alkoxy, halogen-substituted C₂₋₆ alkenyl or halogen-substituted C₂₋₆ alkynyl.

In a preferred embodiment, the present invention provides a compound of formula (IV), wherein an amide bond connection is directly formed by the amino group of melphalan and the carboxyl group terminal of the cell-penetrating peptide derivative:

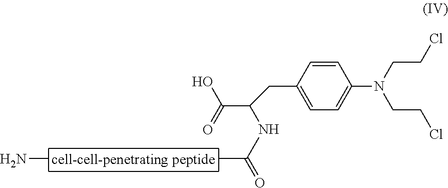

wherein the “cell-penetrating peptide” moiety is as defined for the compound of formula (I), (II) or (III).

In a more preferred embodiment, the present invention provides a compound of formula (V) or a pharmaceutically acceptable salt thereof, which represents a covalent conjugate of cell-penetrating peptide derivatives and melphalan,

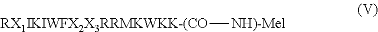

wherein, Mel is the melphalan moiety shown in formula (IV), and the free amino group of Mel forms an amide bond —(CO—NH)— with the carboxyl group of the C-terminal Lys of the cell-penetrating peptide,
X₁, X₂ and X₃ represent hydrophobic amino acids, each of which is independently selected from alanine (A), valine (V), leucine (L), isoleucine (I), proline (P), phenylalanine (F), tryptophan (W), methionine (M) and non-naturally occurring amino acids α-aminobutyric acid, α-aminopentanoic acid, α-aminohexanoic acid and α-aminoheptanoic acid.

In a preferred embodiment, any one of X₁, X₂ and X₃ of the compound of formula (V) is tryptophan.

In another preferred embodiment, any two of X₁, X₂ and X₃ of the compound of formula (V) are tryptophan.

In another preferred embodiment, all of X₁, X₂ and X₃ of the compound of formula (V) are tryptophan.

In a more preferred embodiment, the compound of formula (V) is selected from the following compounds or a pharmaceutically acceptable salt thereof:

| Number | Sequence |
|---|---|
| 2WP-Mel | RWIKIWFQNRRMKWKK-(CO-NH)-Mel |
| 8WP-Mel | RQIKIWFWNRRMKWKK-(CO-NH)-Mel |
| 9WP-Mel | RQIKIWFQWRRMKWKK-(CO-NH)-Mel |
| 28WP-Mel | RWIKIWFWNRRMKWKK-(CO-NH)-Mel |
| 29WP-Mel | RWIKIWFQWRRMKWKK-(CO-NH)-Mel |
| 89WP-Mel | RQIKIWFWWRRMKWKK-(CO-NH)-Mel |
| 289WP-Mel | RWIKIWFWWRRMKWKK-(CO-NH)-Mel |

In a most preferred embodiment, the compound of formula (V) is selected from the following compounds or a pharmaceutically acceptable salt thereof:

| Number | Sequence |
|---|---|
| 89WP-Mel | RQIKIWFWWRRMKWKK-(CO-NH)-Mel |
| 289WP-Mel | RWIKIWFWWRRMKWKK-(CO-NH)-Mel |

### II. Pharmaceutical Compositions

In the second aspect, the present invention provides a pharmaceutical composition comprising a compound of formula (I), (II), (III), (IV) or (V) as described above or a pharmaceutically acceptable salt thereof, and pharmaceutically acceptable excipients or carriers.

In some preferred embodiments, the compound of formula (I), (II), (III), (IV) or (V) is as described in each preferred embodiment of the first aspect.

In some embodiments, the compound of formula (I), (II), (III), (IV) or (V) is formulated in the form of liquid pharmaceutical composition for administration. Available carriers and solvents include water, Ringer's solution, phosphate buffer solution, acetate buffer solution, citrate buffer solution, borate buffer solution, carbonate buffer solution and isotonic sodium chloride solution, glucose solution, etc. In addition, sterile nonvolatile oil can also be used as a solvent or suspending medium of the composition where appropriate. For such purpose, any mixture of nonvolatile mineral or non-mineral oils, including synthetic mono- or diglycerides, may be used. Moreover, fatty acids such as oleic acid can also be used in liquid pharmaceutical compositions.

In a preferred embodiment, the pharmaceutical compositions are eye drops. In the eye drops, the pharmaceutically acceptable carriers are the aqueous carriers that are commonly used in the field of ophthalmic drug, such as sterile water, Ringer's solution, phosphate buffer solution, acetate buffer solution, citrate buffer solution, borate buffer solution, carbonate buffer solution and isotonic sodium chloride solution, glucose solution, etc.

In some embodiments, the pharmaceutical composition comprising a compound of the present invention is a solution or dry powder formulation for injection. For example, the composition is lyophilized powder that can be reconstituted into an injection solution with pharmaceutically acceptable liquid carriers. The pharmaceutically acceptable liquid carriers may be sterile water, Ringer's solution, phosphate buffer solution, acetate buffer solution, citrate buffer solution, borate buffer solution, carbonate buffer solution and isotonic sodium chloride solution, glucose solution, etc.

In some embodiments, the pharmaceutical composition of the present invention is administrated by subcutaneous injection, intramuscular injection, or intravenous injection.

In some embodiments, the pharmaceutical composition of the present invention is administrated by intravenous infusion.

In some embodiments, the pharmaceutical composition of the present invention is administrated by intraocular injection, such as intravitreal injection.

In some embodiments, the pharmaceutical composition of the present invention is administrated by topical administration, such as administrating to the eyes in the form of eye drops.

The liquid pharmaceutical compositions of the present invention are preferably eye drops, such as the liquid formulations formulated in water, Ringer's solution, phosphate buffer solution, acetate buffer solution, citrate buffer solution, borate buffer solution, carbonate buffer solution and isotonic sodium chloride solution, etc. Preferably, the liquid composition of the present invention may comprise (i) a compound of the present invention; (ii) a buffering agent; and (iii) an ophthalmologically acceptable solvent.

In some embodiments, the liquid pharmaceutical compositions comprising a compound of the present invention contain a compound of the present invention at a concentration of 0.001 mg/mL-300 mg/mL, such as 0.01 mg/mL-100 mg/mL or 0.1 mg/mL-50 mg/mL.

Under the concentrations described above, the administration dose of the pharmaceutical composition comprising a compound of the present invention may be 0.1 μL-1000 mL, wherein the administration volume is 0.1 μL-100 μL for single eye dropping, 0.1 μL-100 μL for single intraocular injection, 1 μL-100 mL for single injection, and 0.1 mL-1000 mL for single intravenous infusion.

The administration frequency may be six times a day, three times a day, twice a day, once a day, once every two days, once every three days, twice a week, once a week, once every two weeks, once every four weeks or more time. The administration cycle may be one week, two weeks, three weeks, one month, two months, three months or longer, and the intervals between each administration cycle may be the same or different.

In some embodiments, the compound of the present invention or a pharmaceutical composition thereof can be administered alone or in combination with other drugs.

### III. Use

In the third aspect, the present invention relates to a use of a compound of formula (I), (II), (III), (IV) or (V) or a pharmaceutically acceptable salt thereof in the manufacture of a medicant for preventing or treating ocular diseases in an individual.

In the forth aspect, the present invention relates to a compound of formula (I), (II), (III), (IV) or (V) or a pharmaceutically acceptable salt thereof, for use in preventing or treating ocular diseases in an individual.

In some preferred embodiments, the compound of formula (I), (II), (III), (IV) or (V) is as described in each preferred embodiment of the first aspect.

In some embodiments, the individual is human, such as a child, an adolescent, or an adult.

In some embodiments, the ocular diseases are selected from: tumors of eyelids, conjunctiva, various layer tissues of eyeball (cornea, sclera, uvea and retina) and appendages of the eyes (lacrimal apparatus, orbital and periorbital structures), including malignant basal cell carcinoma, meibomian gland carcinoma, squamous epithelial carcinoma, melanoma, retinoblastoma, choroidal melanoma, rhabdomyosarcoma, lacrimal gland adenocarcinoma, benign choroidal hemangioma, optic nerve glioma, neurofibroma, keratosis, nevus, dermoid tumor, cavernous hemangioma, dermoid cyst, lacrimal gland mixed tumor, and intraocular metastatic carcinoma, particularly retinoblastoma and choroidal melanoma, and also uveitis. Preferably, the ocular diseases are retinoblastoma or choroidal melanoma.

### IV. Method of Treatment

In the fifth aspect, the present invention relates to a method for preventing or treating ocular diseases, comprising administering a compound of formula (I), (II), (III), (IV) or (V) as described in the first aspect or a pharmaceutically acceptable salt thereof, or a pharmaceutical composition as described in the second aspect, to an individual in need thereof.

In some preferred embodiments, the compound of formula (I), (II), (III), (IV) or (V) is as described in each preferred embodiment of the first aspect.

In some embodiments, the individual is human, such as a child, an adolescent, or an adult.

In some embodiments, the ocular diseases are selected from: tumors of eyelids, conjunctiva, various layer tissues of eyeball (cornea, sclera, uvea and retina) and appendages of the eyes (lacrimal apparatus, orbital and periorbital structures), including malignant basal cell carcinoma, meibomian gland carcinoma, squamous epithelial carcinoma, melanoma, retinoblastoma, choroidal melanoma, rhabdomyosarcoma, lacrimal gland adenocarcinoma, benign choroidal hemangioma, optic nerve glioma, neurofibroma, keratosis, nevus, dermoid tumor, cavernous hemangioma, dermoid cyst, lacrimal gland mixed tumor, and intraocular metastatic carcinoma, particularly retinoblastoma and choroidal melanoma, and also uveitis. Preferably, the ocular diseases are retinoblastoma or choroidal melanoma.

In a preferred embodiment, the method of treatment achieves the treatment in an individual by topically administrating a compound of formula (I), (II), (III), (IV) or (V) as described in the first aspect or a pharmaceutically acceptable salt thereof, or a pharmaceutical composition as described in the second aspect, e.g., by administrating to the eyes in the form of eye drops. The eye drops of the present invention may be liquid formulations formulated in water, Ringer's solution, phosphate buffer solution, acetate buffer solution, citrate buffer solution, borate buffer solution, carbonate buffer solution and isotonic sodium chloride solution, etc.

### Definitions

To illustrate the present description, the following definitions will be used, and if appropriate, the terms used in the singular may also include the plural, and vice versa. It should be understood that the terms as used herein are only for the purpose of describing the specific embodiments, and are not intended to be limited.

The terms “halogen” or “halo” as used herein refer to F, Cl, Br or I. Moreover, the term “halogen-substituted” group intends to include both monohalogenated and polyhalogenated group, wherein one or more hydrogens in the group are replaced by one or more same or different halogens.

The term “alkyl” as used herein refers to a saturated straight or branched hydrocarbon group consisitng of carbon atoms and hydrogen atoms. Specifically, the alkyl has 1 to 10, such as 1 to 6, 1 to 5, 1 to 4, 1 to 3 or 1 to 2 carbon atoms. For example, the term “C₁-C₆ alkyl” as used herein refers to a saturated straight or branched hydrocarbon group having 1 to 6 carbon atoms. Examples are, e.g., methyl, ethyl, propyl (including n-propyl and isopropyl), butyl (including n-butyl, isobutyl, sec-butyl or tert-butyl), pentyl (including n-pentyl, isopentyl, neo-pentyl), n-hexyl, 2-methylpentyl, etc. The term “C₁₋₆ alkyl substituted with one or more deuterium” refers to a C₁₋₆ alkyl wherein one or more hydrogen atoms are replaced by isotope deuterium, such as fully deuterated methyl.

The term “alkenyl” as used herein refers to a unsaturated straight or branched hydrocarbon group consisting of carbon atoms and hydrogen atoms and containing at least one double bond. Specifically, the alkenyl has 2 to 8, such as 2 to 6, 2 to 5, 2 to 4 or 2 to 3 carbon atoms. For example, the term “C₂-C₆ alkenyl” as used herein refers to a straight or branched alkenyl having 2 to 6 carbon atoms, such as vinyl, propenyl, allyl, butenyl, pentenyl, etc.

The term “alkynyl” as used herein refers to a unsaturated straight or branched hydrocarbon group consisting of carbon atoms and hydrogen atoms and containing at least one triple bond. Specifically, the alkynyl has 2 to 8, such as 2 to 6, 2 to 5, 2 to 4 or 2 to 3 carbon atoms. For example, the term “C₂-C₆ alkynyl” as used herein refers to a straight or branched alkynyl having 2 to 6 carbon atoms, such as ethynyl, propynyl, propargyl, butynyl, etc.

The term “alkoxy” as used herein refers to the group —O-alkyl, wherein the alkyl moiety is as defined herein. Specifically, said term includes the group —O—C₁₋₆ alkyl, and more specifically —O—C₁₋₃ alkyl. Representative examples of alkoxy include, but are not limited to methoxy, ethoxy, propoxy (including n-propoxy, isopropoxy), butoxy (including n-butoxy, isobutoxy, tert-butoxy), pentyloxy (including n-pentyloxy, isopentyloxy, neopentyloxy), hexyloxy (including n-hexyloxy, isohexyloxy), etc.

The term “halogen-substituted C₁-C₆ alkyl” as used herein refers to the C₁-C₆ alkyl as described above, wherein one or more (e.g., 1, 2, 3, 4 or 5) hydrogen atoms are replaced by halogen atom(s). It should be understood by those skill person that, where there are more than one halogen substituents, the halogens may be the same or different and may be located on the same or different C atoms. Examples of “halogen-substituted C₁-C₆ alkyl” include for example —CH₂F, —CHF₂, —CF₃, —CCl₃, —C₂F₅, —C₂Cl₅, —CH₂CF₃, —CH₂Cl, —CH₂CH₂CF₃ or —CF(CF₃)₂, etc.

The term “halogen-substituted C₂-C₆ alkenyl” as used herein refers to the C₂-C₆ alkenyl as described above, wherein one or more (e.g., 1, 2, 3 or 4) hydrogen atoms are replaced by halogen atom(s). It should be understood by those skill person that, where there are more than one halogen substituents, the halogens may be the same or different and may be located on the same or different C atoms.

The term “halogen-substituted C₂-C₆ alkynyl “as used herein refers to the C₂-C₆ alkynyl as described above, wherein one or more (e.g., 1, 2, 3 or 4) hydrogen atoms are replaced by halogen atom(s). It should be understood by those skill person, where there are more than one halogen substituents, the halogens may be the same or different and may be located on the same or different C atoms.

The term “cycloalkyl” as used herein refers to a monocyclic, fused polycyclic, bridged polycyclic or spiro non-aromatic saturated monovalent hydrocarbon ring structure having the specified number of ring atoms. The cycloalkyl may have 3 to 12 carbon atoms (i.e., C₃-C₁₂ cycloalkyl), such as 3 to 10, 3 to 8, 3 to 7, 3 to 6, 5 to 6 carbon atoms. Examples of suitable cycloalkyl include, but are not limited to monocyclic structure, such as cyclopropyl, cyclobutyl, cyclopentyl, cyclohexyl, cycloheptyl, or cyclooctyl; or polycyclic (e.g., bicyclic) structure, including spiro, fused or bridged system, such as bicyclo[1.1.1]pentyl, bicyclo[2.2.1]heptyl, spiro[3.4]octyl, bicyclo[3.1.1]hexyl, bicyclo[3.1. 1]heptyl or bicyclo[3.2.1]octyl, etc.

The term “cycloalkyl” as used herein further includes “cycloalkenyl”. The “cycloalkenyl” refers to a monocyclic, fused polycyclic, bridged polycyclic or spiro non-aromatic unsaturated hydrocarbon ring structure having the specified number of ring atoms, comprising at least one (e.g., 1, 2, or 3) carbon-carbon double bonds. The cycloalkenyl may have 3 to 12 carbon atoms (i.e., C₃-C₁₂ cycloalkenyl), such as 3 to 10, 3 to 8, 3 to 7, 3 to 6, 5 to 6 carbon atoms. Examples of suitable cycloalkenyl include, but are not limited to monocyclic structure, such as cyclopropenyl, cyclobutenyl, cyclopentenyl, cyclopentadienyl, cyclohexenyl, cyclohexadienyl, cycloheptenyl, cycloheptadienyl, cycloheptatrienyl or cyclooctenyl.

The term “heterocycloalkyl” as used herein refers to a monocyclic, fused polycyclic, spiro or bridged polycyclic non-aromatic saturated ring structure including one or more (e.g., 1, 2, 3 or 4) heteroatoms independently selected from O, N and S and having the specified number of ring atoms, or N-oxides thereof, or S-oxides or S-dioxides thereof. The heterocycloalkyl may have 3 to 12 ring members (may be referred to as a 3- to 12-membered heterocycloalkyl), such as 3 to 10 ring members, 3 to 8 ring members, 3 to 7 ring members, 4 to 7 ring members, 4 to 6 ring members, 5 to 6 ring members. The heterocycloalkyl typically contains at most 4 (e.g., 1, 2, 3 or 4) heteroatoms. Examples of suitable heterocycloalkyl include, but are not limited to azacyclobutyl, oxacyclobutyl, thiocyclobutyl, pyrrolidinyl (e.g., 1-pyrrolidinyl, 2-pyrrolidinyl and 3-pyrrolidinyl), tetrahydrofuranyl (e.g., 1-tetrahydrofuranyl, 2-tetrahydrofuranyl and 3-tetrahydrofuranyl), tetrahydrothienyl (e.g., 1-tetrahydrothienyl, 2-tetrahydrothienyl and 3-tetrahydrothienyl), piperidyl (e.g., 1-piperidyl, 2-piperidyl, 3-piperidyl and 4-piperidyl), tetrahydropyranyl (e.g., 4-tetrahydropyranyl), tetrahydrothiopyranyl (e.g., 4-tetrahydrothiopyranyl), morpholinyl (e.g., morpholino), thiamorpholinyl, dioxanyl, piperazinyl or azacycloheptyl, diazacycloheptyl such as 1,4-diazacycloheptyl, 3,6-diaza-bicyclo[3.1.1]heptyl or 3-aza-bicyclo[3.2.1]octyl. The atoms in the heterocycloalkyl that is attached to the rest of the compound may be carbon atom or heteroatom wherever chemically feasible.

The term “heterocycloalkyl” as used herein further includes “heterocycloalkenyl”, which refers to the “heterocycloalkyl” as defined herein comprising at least one (e.g., 1, 2 or 3) double bonds, such as pyrrolinyl (e.g., 1-pyrrolinyl, 2-pyrrolidinyl, 3-pyrrolinyl, 4-pyrrolinyl or 5-pyrrolinyl), dihydrofuranyl (e.g., 1-dihydrofuranyl, 2-dihydrofuranyl, 3-dihydrofuranyl, 4-dihydrofuranyl or 5-dihydrofuranyl), dihydrothiophenyl (e.g., 1-dihydrothiophenyl, 2-dihydrothiophenyl, 3-dihydrothiophenyl or 4-dihydrothiophenyl), tetrahydropyridinyl (e.g., 1-, 2-, 3-, 4-, 5- or 6-tetrahydropyridinyl), tetrahydropyranyl (e.g., 4-tetrahydropyranyl) or tetrahydrothiopyranyl (e.g., 4-tetrahydrothiopyranyl).

The term “aryl” as used herein refers to a monovalent aromatic hydrocarbonyl derived by removing one hydrogen atom from single carbon atom of aromatic ring system. Specifically, the aryl refers to a monocyclic or fused polycyclic aromatic ring structure having the specified ring atoms. Specifically, said term includes a group comprising 6 to 14, such as 6 to 10, preferbly 6 ring members. Particular aryl includes phenyl and naphthyl, and the most specific aryl is phenyl.

The term “heteroaryl” as used herein refers to a monocyclic or fused polycyclic aromatic ring system having the specified number of ring atoms and including one or more (e.g., 1, 2, 3 or 4) heteroatoms independently selected from O, N and S, or N-oxides thereof, or S-oxides thereof or S-dioxides thereof. Specifically, said aromatic ring structure may have 5 to 10 ring members. The heteroaryl may be, for example, 5- to 6-membered monocycle, or fused bicycle structure formed by two fused 6-membered rings, two fused 5-membered rings, fused 6-membered ring and 5-membered ring, or fused 5-membered ring and 4-membered ring. The heteroaryl ring typically contains up to 4 heteroatoms, more typically up to 3 heteroatoms, more typically up to 2, such as single heteroatom independently selected from O, N and S, wherein N and S may be in oxidation state, such as N oxides, S═O or S(O)₂. In one embodiment, the heteroaryl ring contains at least one ring nitrogen atom, at least one ring sulfur atom or at least one ring oxygen atom. For example, the heteroaryl may be a fused ring comprising 1, 2, 3 or 4 heteroatoms independently selected from N, O or S, such as benzofuran, benzothiophene, indole, benzoimidazole, indazole, benzotriazole, pyrrolo[2,3-b]pyridine, pyrrolo[2,3-c]pyridine, pyrrolo[3,2-c]pyridine, pyrrolo[3,2-b]pyridine, imidazo[4,5-b]pyridine, imidazo[4,5-c]pyridine, pyrazolo[4,3-d]pyridine, pyrazolo[4,3-c]pyridine, pyrazolo[3,4-b]pyridine, isoindole, pyrazolo[3,4-c]pyridine, purine, purrocoline, imidazo[1,2-a]pyridine, imidazo[1,5-a]pyridine, pyrazolo[1,5-a]pyridazine, pyrrolo[1,2-b]pyrimidine, imidazo[1,2-c]pyrimidine, 5H-pyrrolo[3,2-b]pyrazine, 1H-pyrazolo[4,3-b]pyrazine, 1H-pyrazolo[3,4-d]pyrimidine, 7H-pyrrolo[2,3-d]pyrimidine, quinoline, isoquinoline, cinnoline, quinazoline, quinoxaline, phthalazine, 1,6-naphthyridine, 1,7-naphthyridine, 1,8-naphthyridine, 1,5-naphthyridine, 2,6-naphthyridine, 2,7-naphthyridine, pyrido[3,2-d]pyrimidine, pyrido[4,3-d]pyrimidine, pyrido[3,4-d]pyrimidine, pyrido[2,3-d]pyrimidine, pyrido[2,3-b]pyrazine, pyrido[3,4-b]pyrazine, pyrimido[5,4-d]pyrimidine, pyrazino[2,3-b]pyrazine and pyrimido[4,5-d]pyrimidine. For example, the heteroaryl may be a 5- to 6-membered heteroaryl comprising 1 or 2 heteroatoms independently selected from N, O or S. Examples of suitable 5-membered monocyclic heteroaryl include, but are not limited to pyrrolyl, furanyl, thiophenyl, imidazolyl, furazanyl, oxazolyl, oxadiazolyl, oxatriazolyl, isoxazolyl, thiazolyl, isothiazolyl, pyrazolyl, triazolyl or tetrazolyl. Examples of suitable 6-membered monocyclic heteroaryl include, but are not limited to pyridinyl, pyrazinyl, pyridazinyl, pyrimidinyl or triazinyl. The atoms in the heteroaryl that is attached to the rest of the compound may be carbon atoms or heteroatoms wherever chemically feasible.

The substituent that is described as “optionally substituted” means that a group may be unsubstituted or be substituted with one or more (e.g., 0, 1, 2, 3, 4 or 5 or more, or any range that can be derived therefrom) substituents listed for said group, wherein the substituents may be the same or different. In one embodiment, the optionally substituted group is substituted with 1 substituent. In another embodiment, the optionally substituted group is substituted with 2 substituents. In another embodiment, the optionally substituted group is substituted with 3 substituents. In another embodiment, the optionally substituted group is substituted with 4 substituents.

The term “comprising” or “including” as described herein or any variants thereof means the inclusion of said elements, integers or steps, but not excluding any other elements, integers or steps. Herein, when the terms “comprising” or “including” or any variants thereof are used, unless otherwise specified, it also encompasses the situation consisting of said elements, integers or steps. For example, when referring to a polypeptide “comprising” certain specific sequence, it also encompasses the polypeptides consisting of said specific sequence.

The “individual” includes mammal. The mammals include, but are not limit to, domestic animals (e.g., cow, sheep, goat, cat, dog, and horse), primates (e.g., human and non-human primates, such as monkey), rabbits, and rodents (e.g., mice and rats). In some embodiments, the individual is human, including a child, an adolescent, or an adult.

The term “treat”, “treating” or “treatment” as used herein means slowing, interrupting, arresting, alleviating, stopping, reducing, or reversing the progression or severity of an existing symptom, disorder, condition, or disease.

The term “prevent” or “preventing” as used herein includes the inhibition of the occurrence or development of a disease or disorder or a symptom of the specified disease or disorder. In some embodiments, the individuals with a family history of the disease are the candidates for the prophylactic regimens. In general, the term “prevent” or “preventing” refers to the administration of a drug prior to the onset of signs or symptoms, especially in at-risk individuals.

The term “effective amount” refers to an amount of effectively achieving the required treatment outcome at the required dose and with the duration of required period. A therapeutically effective amount of a formulation, compound or composition of the present invention may vary depending on various factors, such as disease state, age, sex and weight of the individual, and the capability of the antibody or antibody part to stimulate a required response in the individual. The therapeutic effective amount is also such an amount that any toxic or detrimental effect of the formulation, compound or composition is lower than the therapeutic beneficial effect.

The “prophylactically effective amount” refers to an amount of effectively achieving the required prophylactic outcome at the required dose and with the duration of required period. In general, a prophylactically effective amount will be lower than a therapeutically effective amount because the prophylactic dose is administered in the subject before or at an earlier stage of the disease.

The term “formulation” or “pharmaceutical composition” as used herein refers to a composition suitable for administration to animals, preferably mammals, including human, comprising at least one active ingredient and at least one inactive ingredient. The formulation of the present invention may be a lyophilized powder formulation or a liquid formulation. The “liquid formulation” or “liquid composition” refers to a formulation in liquid form. The liquid composition of the present invention comprises (i) a compound of the present invention and (iii) a pharmaceutically acceptable liquid carrier.

The “pharmaceutically acceptable carriers” refers to ingredients other than the active ingredient in pharmaceutical formulation, which are nontoxic to the subject. Pharmaceutically acceptable carriers include, but are not limit to, solvents, buffering agents, excipients, stabilizers or preservatives.

The “buffering agent” as used herein refers to a pH buffering agent. For example, the buffering agent is selected from histidine, glutamate, phosphate, acetate, citrate, borate, carbonate and tris-(hydroxymethyl)-aminomethane.

The term “about” when used in conjunction with a numerical value is intended to encompass a numerical value within a range having a lower limit 10% less than the specified numerical value and an upper limit 10% greater than the specified numerical value.

### DESCRIPTION OF DRAWINGS

FIG. 1 is the characterizations of the conjugates Cy5-89WP and 89WP-Mel, wherein: A is the high-performance liquid chromatography (HPLC) spectrum of Cy5-89WP, B is the HPLC spectrum of 89WP-Mel, C is the Mass Spectrometry (MS) spectrum of Cy5-89WP, and D is the Mass Spectrometry spectrum of 89WP-Mel.

FIG. 2 shows the saturated solubility in normal saline of melphalan and the melphalan moiety of the conjugates 89WP-Mel and 289WP-Mel.

FIG. 3 shows the cellular uptake of polypeptide-small molecule conjugates. Comparisons of the flow cytometry diagram and the mean fluorescence intensity of cellular uptake of the conjugates in HCEC and ARPE-19 cells. Administration was performed at a concentration of 3 μM (89WP-FAM, 8.6 μg/mL; Cy5-89WP, 9.7 μg/mL), and incubation was performed at 37° C. for 4 h. One-way ANOVA was used to analyze the significant difference of data, corrected by Tukey's test (n=3, ^{ns}p>0.05, ***p<0.001).

FIG. 4 shows the intracellular distribution of the conjugates 89WP-FAM and Cy5-89WP. The HCEC or ARPE-19 cells were co-incubated with different conjugates at a concentration of 3 μM (89WP-FAM, 8.6 μg/mL; Cy5-89WP, 9.7 μg/mL) at 37° C. for 4 hrs. The cell nuclei were stained with DAPI (blue), and lysosomes were stained with LysoTracker Red DND-99 (the false color of lysosomes was set to red in the 89WP-FAM incubation group, and green in the Cy5-89WP incubation group). Scale bar, 25 μm.

FIG. 5 shows the effects of temperature and endocytosis inhibitors on cellular uptake of 89WP-FAM in ARPE-19 cells. The uptake of 89WP-FAM under the non-inhibited condition was used as control (100%). Administration concentration is 3 μM (8.6 μg/mL). One-way ANOVA was used to analyze the significant difference of data, corrected by Dunnett's test (n=3, ^{ns}p>0.05, ***p<0.001, compared to the control group).

FIG. 6 shows the ability of 89WP to lyse red blood cells at different pH values. Isotonic sodium citrate solution was used as a negative control (hemolysis ratio is 0), and 1 μL of 10% Triton X-100 solution was added to the incubation medium as a positive control (hemolysis ratio is 100%). Two-way ANOVA was used to analyze the significant differences of data, corrected by Tukey's test (n=3, ^{ns}p>0.05, *p<0.05, ***p<0.001, compared with the hemolysis ratio by 89WP at the same concentration at pH 7.4).

FIG. 7 shows the cytotoxicity of the conjugates. The survivals of the HCEC, ARPE-19 and WERI-Rb-1 cells after co-incubation with polypeptides or conjugates were investigated by the MTT or CCK-8 methods (n=4). The HCEC or ARPE-19 cells were co-incubated with polypeptides or conjugates for 4 hrs, followed by incubation in fresh medium for 24 h before addition of MTT for detection. The WERI-Rb-1 cells were co-incubated with the polypeptides or conjugates for 4 hrs, and then CCK-8 was added directly for detection. The cells under normal culture conditions were used as negative control (100% survival).

FIG. 8 is a schematic diagram of an in vitro retinal pigment epithelial cell (ARPE-19 cell) monolayer model.

FIG. 9 shows the distribution of the conjugate 89WP-FAM in ARPE-19 cell monolayer. A is the fluorescence images of ARPE-19 cells at the donor side. B is the permeability depth of 89WP-FAM (green) in the ARPE-19 cell monolayer. The cell nuclei were stained with DAPI (blue). The cell monolayers were co-incubated with 3 μM (8.6 μg/mL) drug-containing solution at 37° C. for 4 hrs. Scale bar: 200 μm.

FIG. 10 shows the penetration behavior of FAM across the ARPE-19 cell monolayer mediated by 89WP. A shows the cumulative permeability amount of FAM or 89WP-FAM at the acceptor side. B shows the apparent permeability coefficient of FAM or 89WP-FAM across the ARPE-19 cell monolayer (Pₐₚₚ, cm·s⁻¹). Administration concentration is 3 μM (89WP-FAM, 8.6 μg/mL). One-way ANOVA was used to analyze the significant difference of data, corrected by Dunnett's test (n=3, ^{ns}p>0.05, ***p<0.001, compared to the FAM group).

FIG. 11 shows the characterizations of 89WP-FAM molecules at the acceptor side.

FIG. 12 shows the uptake of the 89WP-FAM in the WERI-Rb-1 cells at the acceptor side after penetrating across the ARPE-19 cell monolayer. The 89WP-FAM (3 μM, 8.6 μg/mL) and the ARPE-19 cell monolayer were co-incubated at 37° C. for 4 h at the donor side. The WERI-Rb-1 cells were collected at the acceptor side. Flow cytometry diagram (A) and the histogram plot of the mean fluorescence intensity (B) were obtained by flow cytometer. Two-way unpaired t-test was used to analyze the significant difference of data (n=3, ***p<0.001).

FIG. 13 shows the penetration ability of the conjugate Cy5-89WP across the ex vivo rabbit cornea and sclera. A: Frozen sections of cornea and sclera after DAPI staining and Cy5-89WP penetration. B: 2.5D images of Cy5-89WP distribution in sclera. C: Cy5-89WP fluorescence intensity distribution along the white arrows in Figure A. The maximum value of fluorescence intensity was 255. Distances are expressed as percentage of the distance from the arrow origin to the sampling point (i.e., depth of tissue penetration) in the total length of the arrow (i.e., thickness of the whole sclera). Administration concentration is 5 μM (16.2 μg/mL). Scale bar, 100 μm.

FIG. 14 shows the permeability amount of the conjugate Cy5-89WP across the ex vivo cornea and sclera of rabbit. A shows the cumulative permeability amount of Cy5-89WP across the ex vivo tissues. B shows the apparent permeability coefficient of Cy5-89WP across the ex vivo tissues (Pₐₚₚ, cm·s⁻¹). Administration concentration is 5 μM (16.2 μg/mL). One-way ANOVA was used to analyze the significant difference of data, corrected by Tukey's test (n=3, ***p<0.001).

FIG. 15 shows the hematoxylin-eosin staining sections of ex vivo cornea and sclera of rabbit. The tissues were co-incubated with sulfo-Cy5 or Cy5-89WP at 34° C. for 4 hrs. Administration concentration is 5 μM (Cy5-89WP, 16.2 μg/mL). Scale bar, 100 μm.

FIG. 16 shows the distribution of the conjugates 89WP-FAM or Cy5-89WP in the DAPI-stained mice cornea (A) and retina (B) over time. Scale bar, 100 μm. The conjugate 89WP-FAM or Cy5-89WP was dissolved in normal saline at a concentration of 30 μM (89WP-FAM, 85.9 μg/mL; Cy5-89WP, 97.4 μg/mL). The solution (10 μl) was dropped into the conjunctiva sac of the mice. Eyeballs were collected at 0.5, 2, 4, 8, 12, 18 and 24 h after administration. Semi-quantitative results of the mean fluorescence intensity of the distribution of the conjugate 89WP-FAM (C) or Cy5-89WP (D) in the retina (area marked by the orange dotted line) at different time points. The retina sections were divided into five approximate areas from top to bottom, and the mean fluorescence intensity of each area was calculated by ImageJ software. One-way ANOVA was used to analyze the significant difference of data, corrected by Dunnett's test (n=5, ^{ns}p>0.05, **p<0.01, ***p<0.001).

FIG. 17 shows the distribution of 89WP-FAM or Cy5-89WP in mice eyes after eye dropping. Eyeballs were stained with DAPI. The conjugates were dissolved in normal saline at a concentration of 30 μM (89WP-FAM, 85.9 μg/mL; Cy5-89WP, 97.4 μg/mL). The solution (10 μl) was dropped into the conjunctiva sac of the mice. Four hours after dropping, the eyeballs were collected for whole eye sections. Scale bar: 500 μm.

FIG. 18 shows the construction of tumor-bearing mice model and the administration mode.

FIG. 19 shows the immunohistochemical section of brain from the surviving mice in the melphalan solution eye dropping group. The black arrow points to the tumor brain metastasis area. The primary antibody used was against green fluorescent protein in Fluc/GFP-Rb-1 cells. Scale bar: 1 mm.

FIG. 20 shows the hematoxylin-eosin staining sections of mice eyeballs and the comparison of the size of the vitreous seeds after administration. The intraocular tumor regions (vitreous seeds) were outlined with black line, and the area statistics and comparisons were made. Scale bar: 500 μm.

FIG. 21 shows the hematoxylin-eosin staining sections of visceral organs from the mice after administration. Scale bar: 200 μm.

FIG. 22 shows the pharmacodynamics evaluation of the conjugate 89WP-Mel. A: Intraocular tumor proliferation curves of mice in different experimental groups. B: Comparison of intraocular tumor bioluminescence signals among different groups on Day 30 after administration; One-way ANOVA was used to analyze the significant difference of data, corrected by Tukey's test (n=8). C: Comparison of survival curves of mice among different experimental groups. D: Comparison of brain metastases of intraocular tumor of mice among different experimental groups. The administration scheme for retinoblastoma-bearing mice is as follows: normal saline; melphalan solution, 3.0 mg/mL; 89WP-Mel solution 26.2 mg/mL, containing melphalan 3.0 mg/mL; the solution (10 μl) was dropped into the conjunctiva sac of the mice, once a day. 2 μL melphalan solution (0.5 mg/mL) was administrated via intravitreal injection as a positive control, once every two weeks. One-way ANOVA was used to analyze the significant difference of data, corrected by Dunnett's test (^{ns}p>0.05, ***p<0.001).

FIG. 23 shows the brain metastases of intraocular tumor in mice in different administration groups. The letter “T” refers to the areas of tumor growth, and the brown area refers to the green fluorescent protein-positive cells, i.e., the intraocular tumor cells with brain metastasis. Scale bar, 1 mm.

FIG. 24 shows the antiproliferative effect of 89WP-Mel against retinoblastoma. A: Time schedule of treatment and pharmacodynamics evaluation on the mice model. B: Bioluminescence signal intensity of the intraocular tumor of the mice on Day 15 and Day 30 after administration.

FIG. 25 shows the body weight variation of the tumor-bearing mice during the treatment in Section 14 of Example 1. Topi. N.S.: local eye dropping with normal saline, Topi. Mel: local eye dropping with melphalan solution, Topi. 89WP-Mel: local eye dropping with 89WP-Mel solution, IVT. Mel: intravitreal injection with melphalan solution.

FIG. 26 shows the enlarged views of FIG. 23 (the brain metastases of intraocular tumor in mice in different administration groups), wherein the letter T represents the areas of metastatic tumor, the letter P represents strong tumor metastasis (Positive), the letter LP represent the presence of tumor metastasis (Low positive), and the letter N represent no tumor metastasis (Negative).

FIG. 27 shows the routine blood tests of mice in the different groups in Section 14 of Example 1. Topi. N.S.: local eye dropping with normal saline, Topi. Mel: local eye dropping with melphalan solution, Topi. 89WP-Mel: local eye dropping with 89WP-Mel solution, IVT. Mel: intravitreal injection with melphalan solution.

### SPECIFIC EMBODIMENTS

The method of the present invention is further illustrated by the following examples. It should be understood that the following examples are provided merely for the purpose of understanding the present invention, rather than limiting the scope of the present invention in any way.

### Example 1: Construction, Physical and Chemical Characterization, and Pharmacokinetics and Pharmacodynamics Evaluation of Covalent Conjugates of Polypeptide and Small Molecule

### 1. Synthesis and Purification of Polypeptide-Small Molecule Conjugates

The synthesis of polypeptide-small molecule conjugates is shown in Route 1, and the physical and chemical information, such as the molecular weight, of the obtained covalent conjugates is shown in Table 1.

FAM is used as a lipophilic model small molecule. 89WP-FAM is commercially obtained.

Sulfo-Cy5 is used as a hydrophilic model small molecule. The covalent conjugate Cy5-89WP was constructed by maleimide-sulfhydryl reaction. The specific preparation is as follows:

Sulfo-Cy5-Mal (0.35 mg, 1.1 eq.) was dissolved in 5 mL phosphate buffer solution (10 mM PB, pH7.2), and Cys-89WP (1.0 mg, 1.0 eq.) was added under N₂ protection. The reaction was stirred overnight under dark conditions at room temperature. The product was dialyzed (MWCO 2 kD) at 4° C. Cy5-89WP was obtained after a freeze-drying process. In addition, sulfo-Cy5-Mal was capped with Cys, which was used as free small molecule control (sulfo-Cy5) in the subsequent experiments.

The small molecule drug melphalan, which is clinically administrated via intravitreal injection for the treatment of retinoblastoma, is used as a model drug molecule to construct covalent conjugate 89WP-Mel with polypeptides 89WP. The conjugate was prepared by solid phase synthesis techniques, comprising the following three steps:
1) Synthesis of Fmoc-protected melphalan: melphalan (152.6 mg, 2.0 eq.) and 9-fluorenylmethyl-N-succinimidyl carbonate (Fmoc-OSu) (177.0 mg, 2.1 eq.) were mixed in 10 mL dioxane. The reaction was stirred at 50° C. overnight until the system was clear. The solvent was removed under reduced pressure. The product was directly used in the subsequent reaction.
2) Connection of Fmoc-protected melphalan with Wang resin: the product obtained from step 1, DMAP (73.2 mg, 2.4 eq.) and DIC (77.6 μL, 2.0 eq.) were mixed in 5 mL CH₂Cl₂. The mixture was activated for 5 min. Wang resin (0.2273 g, 1.0 eq.) swelled in CH₂Cl₂ for 0.5 hour. Then it was mixed with the above activated product for reaction at 37° C. for 24 h. After the completion of the reaction, the mixture was washed with DMF twice and with CH₂Cl₂ thrice. The CH₂Cl₂ (3 mL), acetic anhydride (120 μL, 5.0 eq.), and pyridine (100 μL, 5.0 eq.) were added subsequently, and the mixture was reacted at 37° C. for 1 hrs. The reaction was washed with CH₂Cl₂ twice and DMF thrice for subsequent use.
3) Extension of peptides chain: The amino acids contained in polypeptide 89WP were attached to the resin from step 2) from the C-terminal to the N-terminal. Firstly, the product from step 2) was mixed with deprotecting agent (20% piperidine solution) and reacted at 37° C. for 15 min. The reaction was carried out twice consecutively. The mixture was washed with DMF for 6 times. Amino acid (8.8 equivalents), condensing agent (HBTU/HOBT) (8.8 equivalents), and DIEA (17.6 equivalents) were added, and the mixture was reacted at 37° C. for 1 hrs. The mixture was washed with DMF for 6 times. The above steps of deprotection-amino acids connection were repeated until the synthesis of the polypeptide was completed. After the last deprotection, the mixture was washed with DMF for 6 times, CH₃OH/CH₂Cl₂ (1/1) for 3 times, and CH₃OH for 3 times. The resin was dried under reduced pressure, and mixed with 4 mL cleavage liquid (TFA: TIS: H₂O=95:2.5:2.5). The reaction was carried out at room temperature for 3 hrs, and then was precipitated in ice diethyl ether, to yield crude 89WP-Mel.

Purification was carried out by preparative liquid chromatography under the following conditions: Preparative column: Waters XBridge™ BEH130 Prep C18 column (19×250 mm, 10 μm); 25-50% acetonitrile gradient, 60 min; flow rate 10 mL/min; column temperature: room temperature; detection wavelength: 214 nm.

The purified product was lyophilized to obtain the final product 89WP-Mel.

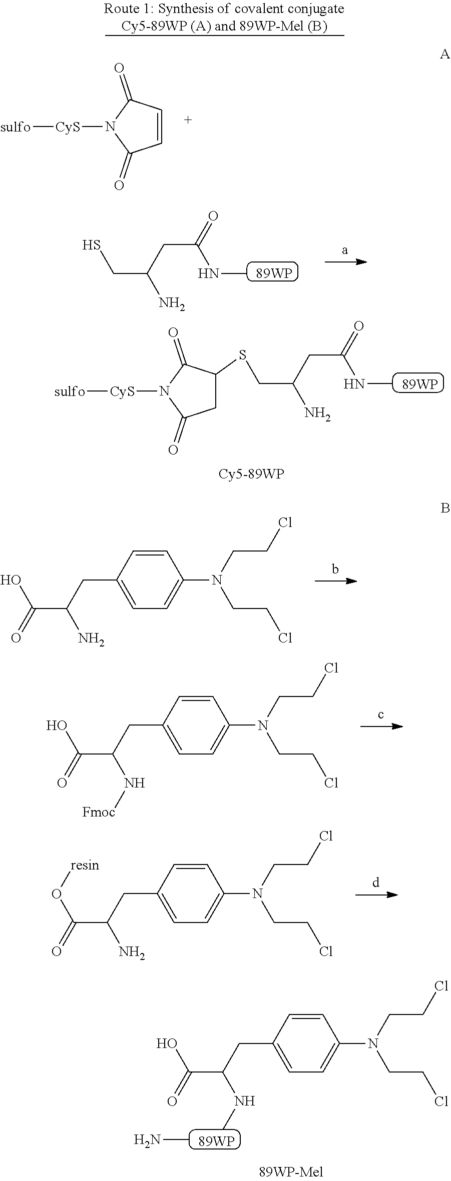

The reaction conditions are as follows: a, stirred overnight at room temperature in 10 mM phosphate buffer in dark under N₂ protection. b, Fmoc-Osu (1.2 equivalents measured by the amounts of melphalan, the same below) is added, and the mixture is stirred in dioxane at 50° C. overnight. c, 1) connection with the resin: 0.5 equivalent of Wang resin, 1.2 equivalents of DMAP and 1 equivalent of DIC are mixed in CH₂Cl₂, and reacted at 37° C. for 24 h; 2) capping the unreacted active sites of the resin: 5.0 equivalents of acetic anhydride and 5.0 equivalents of pyridine are mixed with resin in CH₂Cl₂, and reacted at 37° C. for 1 h; 3) deprotection: 20% piperidine is mixed with resin, and reacted at 37° C. for 15 min; the reaction is repeated twice. d, 1) connection of amino acid: 8.8 equivalents of Fmoc-protected amino acid, 8.8 equivalents of HBTU, 8.8 equivalents of HOBT, and 17.6 equivalents of DIEA are mixed with the deprotected resin in DMF, and are reacted at 37° C. for 1 h; 2) amino acid deprotection: 20% piperidine is mixed with resin, and is reacted at 37° C. for 15 min; the reaction is repeated twice; wherein d1) and d2) are repeated until the synthesis of the polypeptide was completed for subsequent cleavage; 3) cleavage: the resin is mixed with cleavage liquid (TFA:TIS:H₂O=95:2.5:2.5), and the reaction is performed at room temperature for 3 h.

TABLE 1
Physical and chemical information of the polypeptide
89WP and the covalent conjugate molecules

| | | miLogP¹, Mw (Da) of |
|---|---|---|
| | Molecular Weight (Da) | conjugated molecules |
| 89WP | 2376.95 | — |
| 89WP-FAM | 2863.47 | 0.56, 504.52 (Lys-FAM) |
| Cy5-89WP | 3245.21 | −0.58, 886.26 (sulfo-Cy5-Cys) |
| 89WP-Mel | 2664.15 | 0.08, 305.20 (Mel) |

Note
¹https://www.molinspiration.com/cgi-bin/properties

### 2. Characterization of Polypeptide-Small Molecule Conjugates

### 2.1 Characterization of Purity

Purity of the conjugates was characterized by High Performance Liquid Chromatography (HPLC) under the following conditions: Column: YMC-Pack ODS-A column (150×4.6 mm, 5 μm); mobile phase: 5-65% acetonitrile (with 0.1% TFA), 30 min; flow rate: 0.7 mL/min; column temperature: room temperature; detection wavelength: 214 nm; injection volume: 10 μL.

### 2.2 Characterization of Molecular Weight

The molecular weight of the conjugates was characterized by mass spectrometry under the following conditions: Mobile phase: methanol:pure water:formic acid=80:19.9:0.1; flow rate: 0.3 mL/min; capillary voltage: 3000 V; flow rate of drying gas: 12 L/min, temperature: 350° C.

### 2.3 Purity and Molecular Weight of the Polypeptide-Small Molecule Conjugates

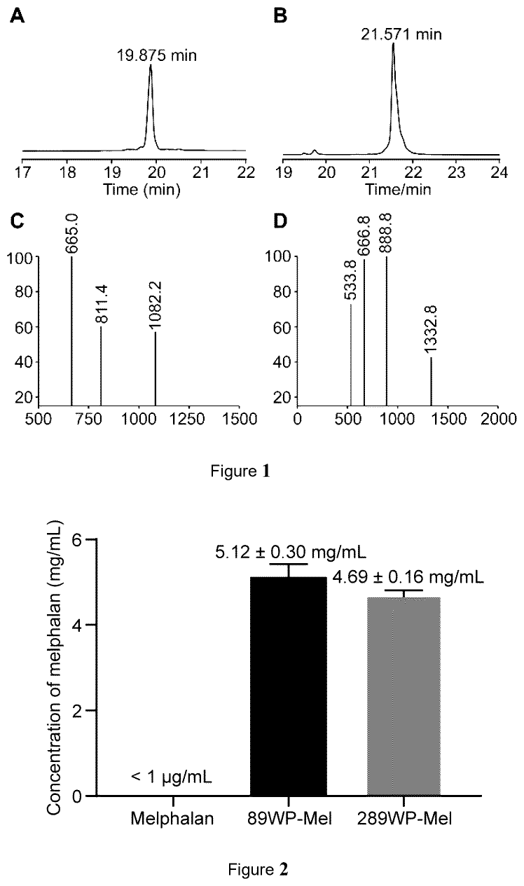

The conjugate 89WP-FAM was obtained commercially. The purity thereof is ≥95%, and the molecular weight is correct. The characterization results of the conjugates Cy5-89WP and 89WP-Mel molecules are shown in FIG. 1. The HPLC results show that the liquid chromatography purity of the two conjugates were both above 95%; the theoretical molecular weight of conjugate Cy5-89WP is 3245.21 (the water-soluble group in the Cy5 molecule is the sulfonic acid group), and the theoretical molecular weight of 89WP-Mel is 2664.15; mass spectrometry results confirmed that the molecular weight is correct.

### 3. Investigation of the Solubilization Ability of Polypeptide-Small Molecule Conjugates

### 3.1 Solubility Assay of Melphalan

Liquid chromatography detection conditions for melphalan: column: YMC-Pack ODS-A column (150×4.6 mm, 5 μm); mobile phase: 40% acetonitrile solution; flow rate: 0.7 mL/min; column temperature: 25° C.; detection wavelength: 260 nm; injection volume: 10 μL.

Solubility assay: melphalan 0.1 mg was accurately weighted and dispersed in 1 mL normal saline, ultrasonicated for 5 min, incubated at 37° C. for 24 hrs, and filtered through 0.22 μm filter membrane. The peak area of the sample was obtained by liquid chromatography. The concentration of the sample was calculated by substituting the peak area in the standard curve equation.

### 3.2 Solubility Assay of Polypeptide-Melphalan Conjugates

Liquid chromatography detection conditions for 89WP-Mel and 289WP-Mel: column: YMC-Pack ODS-A column (150×4.6 mm, 5 μm); mobile phase: 5-65% acetonitrile (with 0.1% TFA), 30 min; flow rate: 0.7 mL/min; column temperature: 25° C.; detection wavelength: 214 nm; injection volume: 10 μL.

Solubility assay: The excess amount of 89WP-Mel or 289WP-Mel was dispersed in 60 μL normal saline, ultrasonicated for 5 min, incubated at 37° C. for 24 hrs, filtered through 0.22 μm filter membrane, and then diluted 10 times and 20 times respectively. The peak area of the samples was obtained by liquid chromatography. The concentration of the samples was calculated by substituting the peak area in the standard curve equation.

### 3.3 Solubilization Ability of Polypeptide-Small Molecule Conjugates

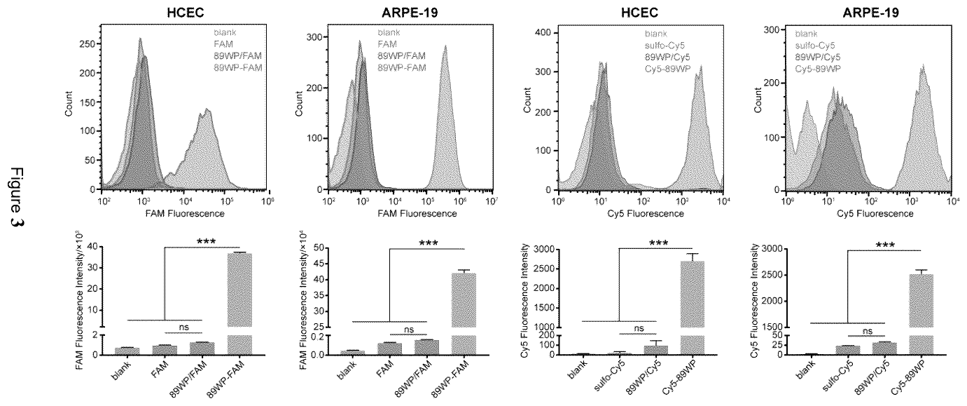

As shown in FIG. 2 and Table 2, the solubility of melphalan in normal saline is lower than 1 μg/mL. When melphalan and polypeptide together formed covalent conjugate 89WP-Mel or 289WP-Mel, the solubility of melphalan moiety may reach 5.12±0.30 mg/mL or 4.69±0.16 mg/mL respectively, which is more than 4500 times higher than that of the original melphalan.

The samples were incubated in normal saline at 37° C. and 160 rpm for 24 h to obtain the saturated solutions. The solubility of the conjugates is converted to the solubility of melphalan moiety.

TABLE 2
Solubility of drug-polypeptide conjugates

| | | | | | | Solubility of | Converted to | times of |
|---|---|---|---|---|---|---|---|---|
| | Drug | Molecular | Polypeptide | Molecular | Solubility | drug-polypeptide | solubility of | increased |
| | name | weight | name | weight | of drug | conjugate | the drug | permeation |
| 1 | melphalan | 305.2 | 89WP | 2376.97 | <1 | 44.69 ± 2.62 | 5.12 ± 0.30 | >5000 |
| | | | | | μg/mL | mg/mL | mg/mL | |
| 2 | melphalan | 305.2 | 289WP | 2435.05 | <1 | 41.80 ± 1.39 | 4.69 ± 0.16 | >4670 |
| | | | | | μg/mL | mg/mL | mg/mL | |

### 4. Cellular Uptake of Polypeptide-Small Molecule Conjugates

The HCEC and ARPE-19 cells with good growth status in the logarithmic phase were placed on 24-well plates at a density of 1×10⁴ cells/well and incubated for 24 hrs. The drug solutions (all comprising 3 μM fluorescein) were added and incubated at 37° C. for 4 hrs. Then, the drug solutions were discarded. After being washed with cold 10 mM PBS (containing 0.02 mg/mL heparin sodium) thrice and trypsinization, the cells were resuspended in 10 mM PBS. The positive cell ratio and mean fluorescence intensity were tested by flow cytometer (FAM, Ex 488 nm/Em 520 nm; Cy5, Ex 638 nm/Em 660 nm).

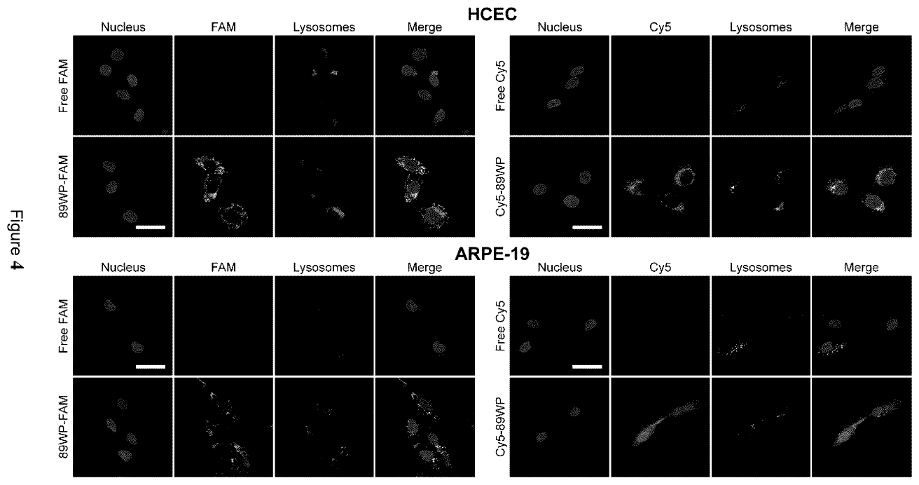

As shown in FIG. 3, the cellular uptake ratios of the covalent conjugates in the two cells were approximately 100%. For 89WP-mediated FAM cellular uptake behavior, the mean fluorescence intensity of the covalent conjugate (89WP-FAM) group was 37 times higher than that of the free FAM group in HCEC cells (p<0.001), and was 342 times in ARPE-19 cells (p<0.001). There is no significant difference between the physical mixture (89WP/FAM) group and the free FAM group (p>0.05). For 89WP-mediated sulfo-Cy5 cellular uptake behavior, the mean fluorescence intensity of the covalent conjugate (Cy5-89WP) group was 125 times higher than that of the free sulfo-Cy5 group in HCEC cells, and was 105 times in ARPE-19 cells. Similarity, there is no significant difference between the physical mixture (89WP/sulfo-Cy5) group and the free sulfo-Cy5 group (p>0.05).

### 5. Intracellular Distribution of Polypeptide-Small Molecule Conjugates

The HCEC and ARPE-19 cells with good growth status in the logarithmic phase were placed in 4-chamber confocal dishes at a density of 1×10⁴ cells/well and incubated for 24 hrs. The drug solutions (all comprising 3 μM fluorescein) were added and incubated at 37° C. for 3.5 hrs. LysoTracker was then added and incubated for 0.5 hrs. Then, the drug solutions were discarded. After being washed with cold 10 mM PBS (containing 0.02 mg/mL heparin sodium) thrice and fixed with 4% paraformaldehyde, the nuclei were stained with DAPI. The fluorescence signal distribution was observed with the laser confocal microscope.

The results of the intracellular distribution of conjugates observed with the laser confocal microscope are shown in FIG. 4. After incubation at 37° C. for 4 hrs, there was no obvious green fluorescence signal in the cells of the free FAM group. However, after co-incubation with 89WP-FAM, there was obvious green fluorescence signal in the HCEC and ARPE-19 cells. Meanwhile, the majority of the green fluorescence was not co-localized with lysosomes labeled by red fluorescence. Similar results were observed for the fluorescein Cy5 and Cy5-89WP after co-incubation with cells. The above results show that, compared with the free fluorescein, the construction of covalent conjugates with polypeptide 89WP can effectively promote the cellular uptake of the fluorescein, and the constructed conjugates have the endosomal escape ability.

### 6. Cellular Uptake Mechanism of Polypeptide-Small Molecule Conjugates

The ARPE-19 cells with good growth status in the logarithmic phase were placed on a 24-well plate at a density of 1×10⁴ cells/well and incubated for 24 hrs. The inhibitor solution was added and incubated at 37° C. or 4° C. for 0.5 hrs. The drug solutions (all comprising 3 μM FAM) were added while the inhibitor was retained. The plate was incubated at 37° C. or 4° C. for 1.5 hrs. The drug solutions were discarded. After being washing with cold 10 mM PBS (containing 0.02 mg/mL heparin sodium) thrice and trypsinization, the cells were resuspended in 10 mM PBS. The positive cell ratio and mean fluorescence intensity were tested by flow cytometer (Ex 488 nm/Em 520 nm).

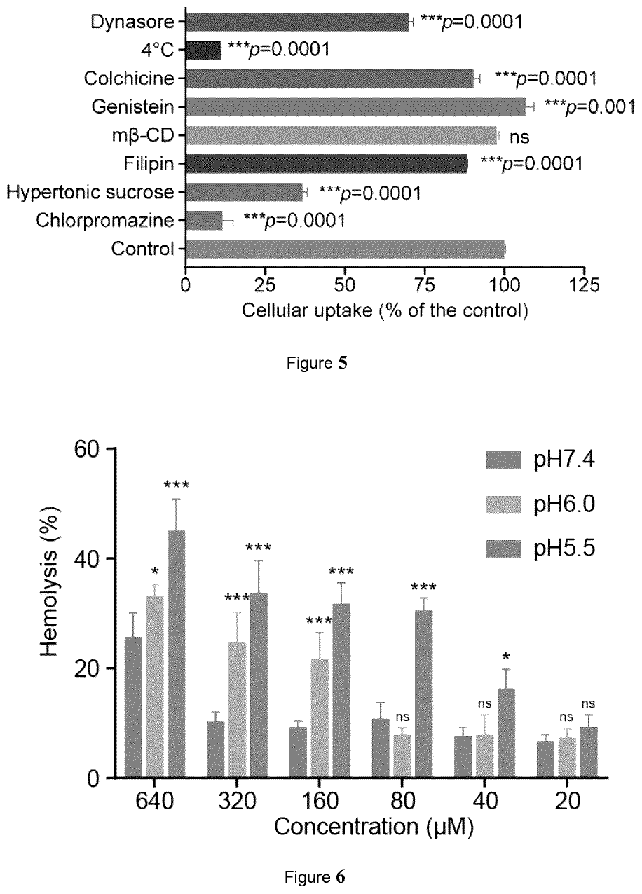

As shown in FIG. 5, the uptake of 89WP-FAM in ARPE-19 was only 1/10 of that of the control group at low temperature; the cellular uptake of 89WP-FAM decreased significantly upon co-incubation with the inhibitor Dynasore (which inhibits the GTPase activity of dynein), hypertonic sucrose or chlorpromazine. In summary, 89WP-FAM enters the ARPE-19 cells mainly through the energy-dependent and clathrin-mediated endocytosis pathway.

### 7. Ability of Polypeptide-Small Molecule Conjugates to Lyse Red Blood Cells

The red blood cells were resuspended at a density of 7×10⁷ cells/mL in incubation medium at different pHs. The cell suspension (75 μL) was mixed with 75 μL of aqueous solution of test sample. Pure water was set as a negative control, and 1 μL Triton X-100 dissolved in 74 μL pure water was set as a positive control. Incubation was performed at 37° C. for 1 h in an air shaker at 200 rpm. After incubation, the samples were centrifugated at 1000 g for 5 min. The supernatant (100 μL) was taken to measure the OD_{450 nm/750 nm} via microplate reader.

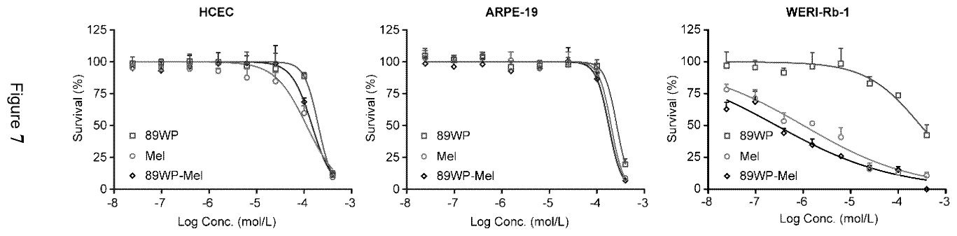

As shown in FIG. 6, at pH 7.4, the hemolysis ratio is around 10% for 89WP at low concentration (<320 μM), and is relatively high only at high concentration (640 μM), being 25.8%. Compared with the condition of pH 7.4, the hemolysis ability significantly increased for 89WP at a concentration of 160 μM at pH 6.0 (p<0.001), which was 21.7%. With the increase of the concentration, the hemolysis ability further increased, and the hemolysis ratio is 33.3% at a concentration of 640 μM. At pH 5.5, the hemolysis ability of 89WP at a concentration of 40 μM (which is 16.4%) showed a significant difference compared with the ability at pH 7.4 (p<0.05). With the increase of the concentration, the hemolysis ability further increased. The hemolysis ratio was 45.1% at a concentration of 640 μM. In summary, the hemolysis ability of 89WP is relatively weak at pH 7.4; as the pH decreases, the hemolysis ability of 89WP significantly increases, and a relatively high hemolysis ratio can be achieved at a lower concentration.

### 8. Cytotoxicity of Polypeptide-Small Molecule Conjugates

The ARPE-19 and WERI-Rb-1 cells with good growth status in the logarithmic phase were placed on 96 well plates at a density of 2000 cells/well. After incubation for 24 hrs, the drug solutions at different concentrations were added. The ARPE-19 cells were incubated at 37° C. for 4 hrs. Then, the drug solutions were discarded. The plates were washed with PBS thrice. The fresh medium was then added and incubated for 24 h. Then, 0.5 mg/mL MTT was added and incubated at 37° C. for 4 hrs. The culture solution was discarded. To each well DMSO (150 μl) was added, and the plates were shaken on a shaker for 20 min. Then, the OD₄₉₀ₙₘ value was measured. For the WERI-Rb-1 cells, they were incubated with drug solutions for 4 hrs, and CCK-8 reagent (10 μL) was added to each well. The plates were incubated at 37° C. for 2 hrs. The OD₄₉₀ₙₘ value was measured directly. In addition, zero_adjustment wells and negative control wells (cell survival rate 100%) were set.

As shown in FIG. 7 and Table 3, the half inhibitory concentration (IC₅₀) of the polypeptide 89WP against the three cells were around 200-300 μM. The IC₅₀ of free melphalan against HCEC and ARPE-19 cells were around 100-200 μM, while the IC₅₀ against WERI-Rb-1 cells was 1.02 μM. The IC₅₀ of the conjugate 89WP-Mel against HCEC and ARPE-19 cells were around 150 μM, while the IC₅₀ against WERI-Rb-1 cells was 0.28 μM. Based on the above results, for the given concentration range (less than 100 μM), 89WP alone has no obvious toxic effect on both normal cells and tumor cells of the eyes, while the conjugate 89WP-Mel has a better inhibitory effect on tumor cell proliferation than free melphalan; moreover, for a certain concentration range (e.g., 1-10 μM), the conjugate can effectively inhibit the proliferation of tumor cells without causing toxic effects on normal cells of eyes.

TABLE 3
The IC₅₀ values of the polypeptide 89WP, melphalan (Mel)
and the conjugate 89WP-Mel against different cells

Compound

| Cell | 89WP | Mel | 89WP-Mel |
|---|---|---|---|
| HCEC | 199.8 μM | 112.8 μM | 146.9 μM |
| ARPE-19 | 263.7 μM | 200.4 μM | 177.4 μM |
| WERI-Rb-1 | 296.9 μM | 1.02 μM | 0.28 μM |

### 9. Penetration Experiment of the Conjugates Across the Retinal Pigment Epithelial Cell Monolayer Model

### 9.1 Construction of Model

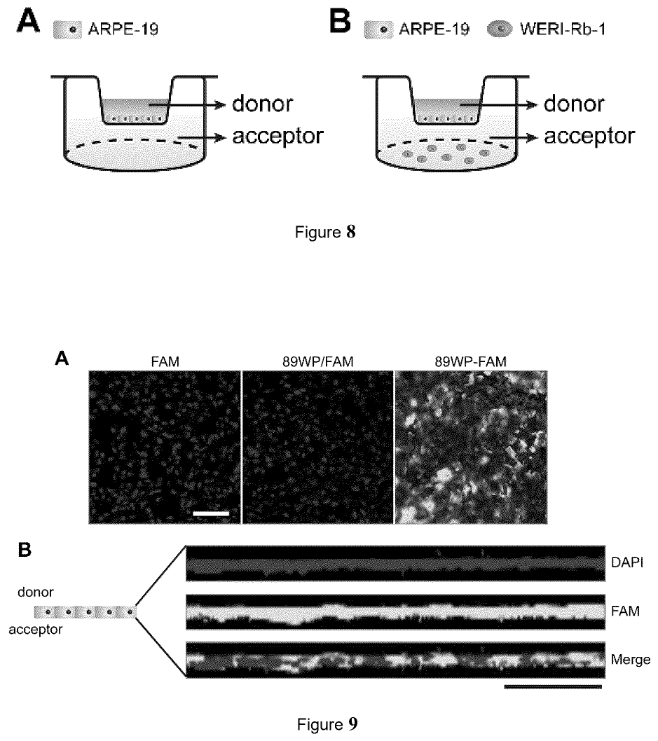

The Corning Transwell Polyester (PET) membrane (diameter of the nested chamber is 6.5 mm, and diameter of the pore is 0.4 μm) was selected as the basement membrane, and coated with rat tail collagen type I. The ARPE-19 cells in good growth state were placed at the upper side of the membrane at 1×10⁴ cells/well, and continuously cultured until the transmembrane resistance reaches the standard (˜100Ω·cm²). Then, the model in FIG. 8A was obtained. The model in FIG. 8B is obtained by seeding the WERI-Rb-1 cells in good growth state at 2×10⁴ cells/well in the lower chamber 24 hours before administration.

### 9.2 Penetration Experiments of the Conjugates Across the Barrier Models

After the construction of the model, the model in FIG. 8A was taken. 100 μL solution of free FAM, a physical mixture of 89WP and FAM (89WP/FAM), or the covalent conjugate (89WP-FAM) (each containing 3 μM FAM) was added to the upper chamber, and 600 μL D-Hank's balanced salt solution as blank solvent was added to the lower chamber. Samples (200 μL for each sampling) were taken every 0.5 h from the lower chamber, and 200 μL fresh D-Hank's balanced salt solution was added immediately. A total of 8 samples were taken. The fluorescence signal intensity of the samples was detected with a microplate reader (Ex 490 nm/Em 520 nm). In addition, the supporting membrane was carefully cut off. The RPE side was washed three times with PBS solution comprising 0.02 mg/mL heparin sodium, fixed with 4% paraformaldehyde, and stained with DAPI. The distribution of fluorescence signal was observed with the laser confocal microscope. In addition, a certain amount of the samples from the lower chamber of the 89WP-FAM group were dialyzed in dark at 4° C. for 24 hours. The resulted samples were subjected to HPLC and MS. The process was the same as that in 2.2 of the first section.

The calculation formula of the Apparent Permeability Coefficient (Pₐₚₚ) is as follows:

$${P}_{app} = Δ Q / ( Δ t \cdot {C}_{0} \cdot A \cdot 3600 )$$

wherein, ΔQ/Δt is the change of the molar amount of the polypeptide across the retinal pigment epithelial cell monolayer model per unit time, which can be obtained from the slope by fitting the cumulative permeability and the diffusion time; C₀ is the initial concentration of the polypeptide in the donor cell, which is 3 μM; A is the area of the cornea or sclera exposed to the diffusion medium, i.e., the effective diffusion area, which is 0.825 cm².

The model in FIG. 8B was subjected to the similar penetration experiment, except that the sampling operation was cancelled. After incubation for 4 hrs, the cells in the lower chamber were directly resuspended in PBS for flow cytometer (Ex 488 nm/Em 520 nm).

### 9.3 Penetration of the Conjugates Across the Retinal Pigment Epithelial Cell Monolayer Model

The distribution of FAM or 89WP-FAM in the retinal pigment epithelial cell monolayer was observed with the laser confocal microscope, and the results are shown in FIG. 9. Four hours after administration, there was no green fluorescence signal at the donor side of the cell monolayer treated with free FAM or with the physical mixture of 89WP and FAM (89WP/FAM), suggesting that the FAM itself or the FAM molecules that are physically mixed with 89WP have difficulty in entering the ARPE-19 cells, which is consistent with the cellular uptake results. However, there was an obvious green fluorescence signal at the donor side of the cell monolayer treated with 89WP-FAM. Moreover, the results of z-axis scanning showed (FIG. 9B) that the fluorescence signal of 89WP-FAM diffused across the whole cell layer, showing that 89WP-FAM could effectively penetrate the cell monolayer.

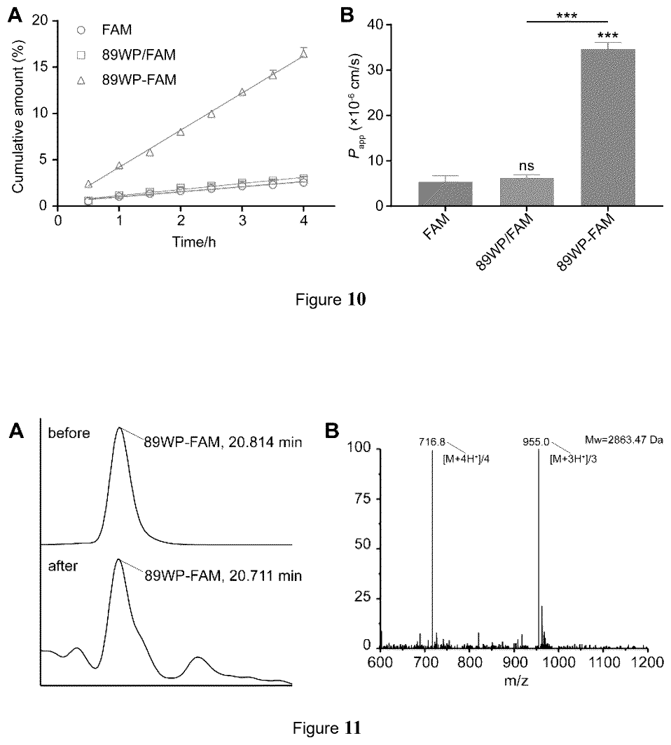

The permeability amount of FAM or 89WP-FAM were compared among the different experimental groups and the results were shown in FIG. 10. Compared with the free FAM, the permeability amount of FAM of the physical mixture with 89WP across the cell monolayer did not significantly increase, and there was no significant difference for the Pₐₚₚ values (p>0.05). However, the permeability amount of the conjugate 89WP-FAM across the cell monolayer significantly increased in a time-dependent manner. The cumulative permeability amount for 4 hours reached 16.5%, which was 6.3 times that of the free FAM group, and the Pₐₚₚ value was 6.4 times that of the free FAM group (p<0.001).

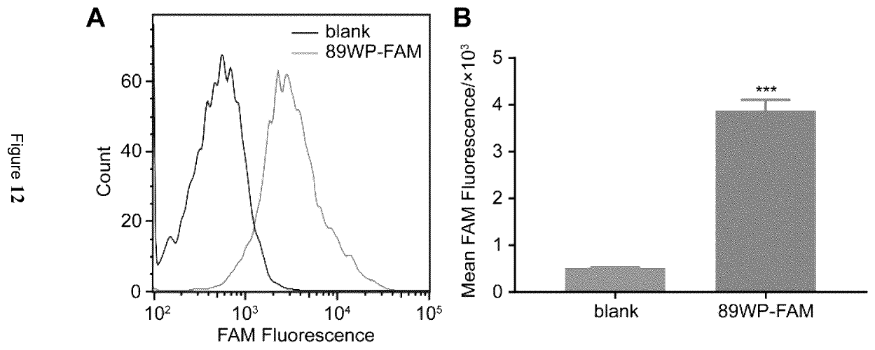

The samples were subjected to liquid chromatography and mass spectrometry after the penetration in the 89WP-FAM group, and the results were shown in FIG. 11. The liquid chromatography retention time of the samples at the accepter side were consistent with that of the original 89WP-FAM. The samples were collected and were subjected to mass spectrometry analysis, showing the same molecular weight with that of 89WP-FAM. Thus, it is confirmed that 89WP-FAM could penetrate the ARPE-19 cell monolayer in an intact form.

The WERI-Rb-1 cells were seeded at the acceptor side to investigate the ability of 89WP-FAM molecules to enter the cells after penetrating across the ARPE-19 cell monolayer, and the results were shown in FIG. 12. Compared with the blank cells, the mean fluorescence intensity of WERI-Rb-1 cells in 89WP-FAM group significantly increased (p<0.001).

### 10. Penetration Experiment of the Conjugates Across the Ex Vivo Cornea and Sclera of Rabbit

The New Zealand male rabbits (1.5 kg) were euthanized with an overdose of anesthesia (amobarbital sodium, 150 mg/kg). The cornea (with 2 mm sclera ring) and sclera were removed immediately and placed vertically in the horizontal diffusion cells, with the epithelium orienting to the donor cells. The small molecule sulfo-Cy5 or conjugate Cy5-89WP solution (3.5 mL, the concentration of sulfo-Cy5 is 5 μM) was added to the donor cells, with Ringer's solution as the diffusion medium. To the acceptor cells 3.5 mL blank Ringer's solution was added, maintaining a constant temperature water bath at 34±0.5° C. Samples (0.5 mL) were taken every 0.5 hour, and 0.5 mL fresh blank Ringer's solution was added immediately. A total of 8 samples were taken. The fluorescence signal intensities of the samples were detected with a microplate reader (Ex 640 nm/Em 680 nm). In addition, at the end of the experiments, the cornea or sclera from the diffusion surface was taken, washed with normal saline for 3 times, fixed with 4% paraformaldehyde, and dehydrated with 30% sucrose overnight. Then, they were made into frozen sections, and were subjected to the laser confocal microscope after DAPI staining. Hematoxylin-eosin (HE) stained paraffin sections were made and subjected to an inverted fluorescence microscope.

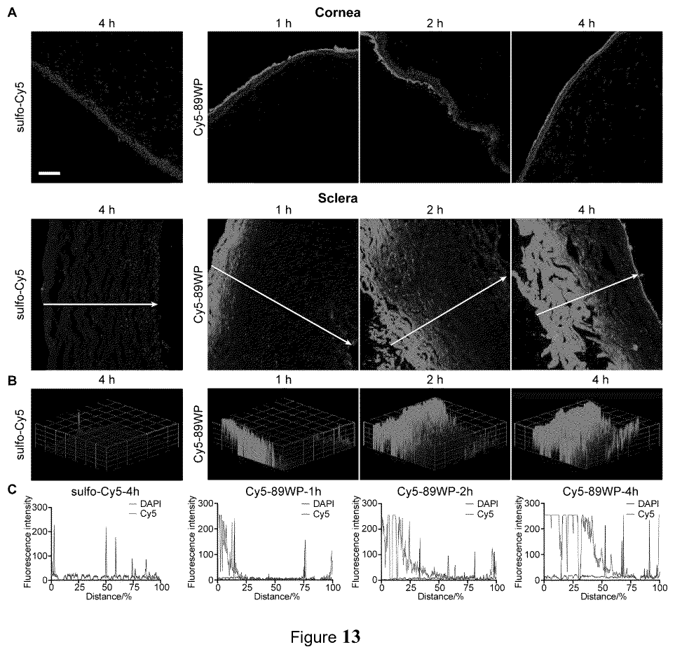

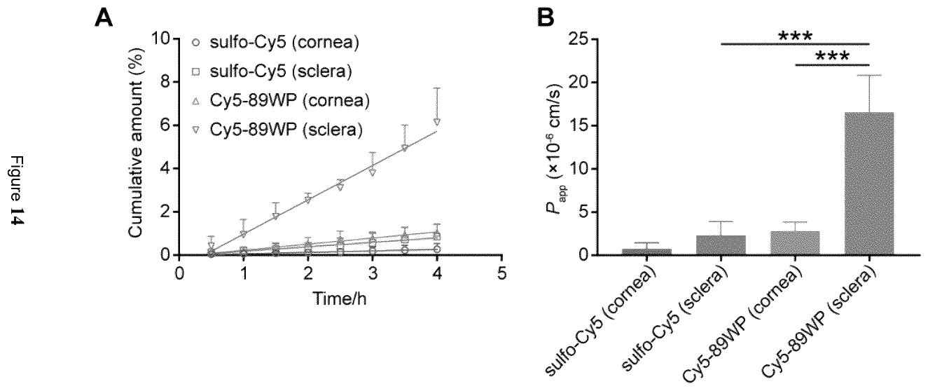

The permeability behavior of the covalent conjugate Cy5-89WP across the tissue was investigated through the ex vivo rabbit cornea and sclera diffusion experiment in combination with the fluorescence method. As shown in FIG. 13, for the ex vivo rabbit cornea, there was almost no red fluorescence signal in the free sulfo-Cy5 group 4 hrs after administration, while there was strong red fluorescence distributed in the corneal epithelium in the Cy5-89WP group from 1 hrs, 2 h up to 4 h after administration. For the ex vivo rabbit sclera, there was a weak red fluorescence signal at the donor side in the free sulfo-Cy5 group 4 hours after administration, while there was a very strong red fluorescence signal along the direction from the donor side to the acceptor side in the Cy5-89WP group 1 hour after administration. Moreover, in the Cy5-89WP group, the permeability depth in the sclera from the donor side to the acceptor side gradually increased over time, and the red fluorescence signal was almost distributed throughout the sclera layer 4 hours later.

The permeability amounts of the free sulfo-Cy5 molecule and the conjugate Cy5-89WP across the ex vivo rabbit cornea and sclera were compared, and the results were shown in FIG. 14. The molecules penetrated the ex vivo cornea and sclera in a linear time-dependent manner. Four hours after administration, the cumulative permeability amount of sulfo-Cy5 across the cornea was only 0.27% of the total exposure, and the cumulative permeability amount across the sclera was only 0.85%. The cumulative permeability amount of Cy5-89WP across the corneal was 1.04%, which is 3.8 times that of sulfo-Cy5, and the cumulative permeability amount across the sclera was 6.13%, which is 7.2 times that of sulfo-Cy5. The Papp value of the free sulfo-Cy5 was (7.3±0.6)×10⁻⁷ cm·s⁻¹ for the cornea, and (2.3±1.3)×10⁻⁶ cm·s⁻¹ for sclera. The Pₐₚₚ value of Cy5-89WP was (2.8±0.9)×10⁻⁶ cm·s⁻¹ for the cornea, which is 3.8 times that of sulfo-Cy5. Particularly, the apparent permeability coefficient (Pₐₚₚ) value of Cy5-89WP was (1.7±0.4)×10⁻⁵ cm·s⁻¹ for the sclera, which is 7.4 times the Pₐₚₚ value of sulfo-Cy5 for the sclera (p<0.001) and is 6.1 times the Pₐₚₚ value of Cy5-89WP for the cornea (p<0.001), suggesting that Cy5-89WP has a stronger permeability ability across the sclera.

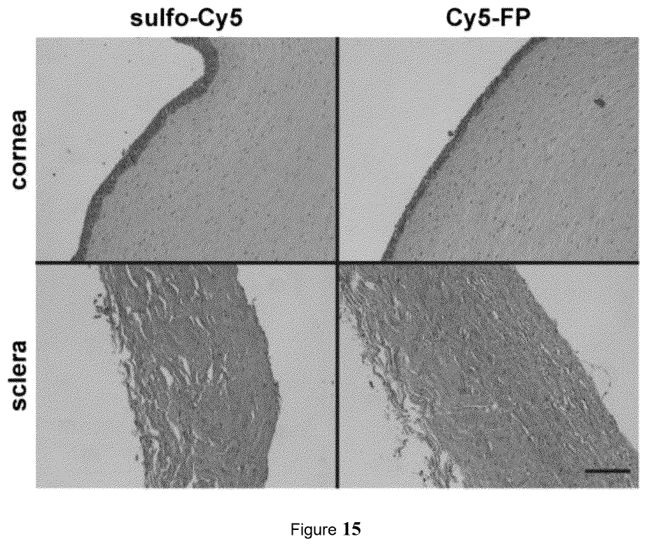

The HE-stained sections of the ex vivo rabbit cornea and sclera after administration were shown in FIG. 15. The structures of cornea and sclera are intact, without fiber breakage. There is no edema in the corneal epithelium, and the sclera structure is dense. It is shown that the conjugates have no toxic effect on the ex vivo rabbit cornea and sclera under the experimental conditions.

### 11. Ocular Pharmacokinetic Evaluation of the Conjugates

The distribution and elimination behavior of the conjugates in the eye tissues was investigated by dropping the drugs into the conjunctival sac of the mice. The conjugate solution 10 μL (at a concentration of 30 μM) was dropped into the conjunctival sac of the mice. The eyelids were gently closed to make the solution distributed evenly. The mice were euthanized by an overdose of anesthesia (amobarbital sodium, 150 mg/kg) at the corresponding time points (30 min, 2 h, 4 h, 8 h, 12 h, 18 h, 24 h). The eyeballs were taken out and fixed with FAS eyeball fixative solution for 24 hours, dehydrated with 15% and 30% sucrose solution gradients, and then embedded in OCT to prepare 8 μm thick eyeball frozen sections. The cell nuclei were stained with DAPI. The distribution of the conjugates in anterior ocular segment (cornea) and posterior ocular segment (retina) was observed with the laser confocal microscope.

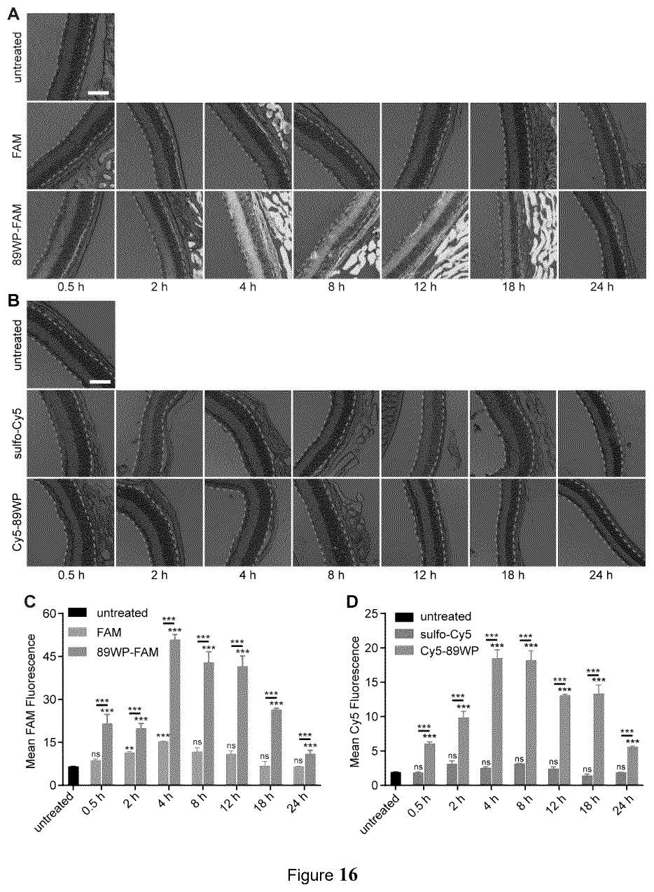

The intraocular distribution behavior of the conjugates after dropping was observed by eye sections of the mice. As shown in FIGS. 16A and 16B, there was no FAM or Cy5 fluorescence background signal in the blank retinal sections; for lipophilic FAM molecules, there was a weak green fluorescence signal in the retina from 2 h to 12 h after eye dropping. The semi-quantitative results showed (FIG. 16C) that the fluorescence intensity of the free FAM in the retina reached the peak 4 hours after administration, and there was no significant difference in the fluorescence intensity between the retina treated with free FAM and the blank retina 8 hours later, suggesting that the elimination was basically completed. For the hydrophilic sulfo-Cy5 molecule, there was no significant difference in the fluorescence intensity between the retina after administration and the blank retina (FIG. 16D), suggesting that it is difficult to be absorbed into the retina from the ocular surface. The retinal fluorescence intensities in the 89WP-FAM group and the Cy5-89WP group were both significantly stronger than that in the corresponding free molecule group at the same time point after administration. The retinal fluorescence intensity in the covalent conjugate group reached the peak at about 4 hours after administration, and thereafter the fluorescence intensity decreased gradually. However, the retinal fluorescence intensity in the conjugate group was still significantly higher than that in the blank retina after 24 h.

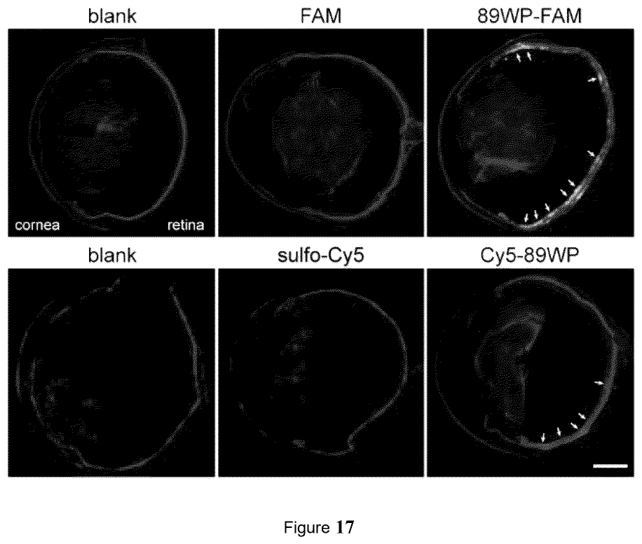

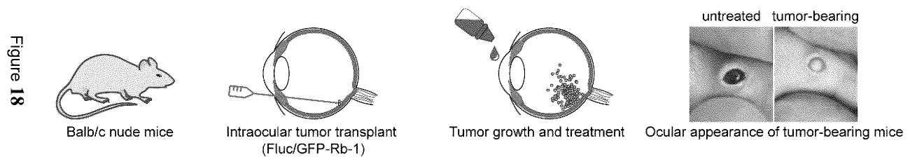

The ocular distribution area of the conjugates after administration was observed with the whole eye sections. As shown in FIG. 17, there was no fluorescence background signal in the blank eyes. For FAM or sulfo-Cy5 molecules, there was no obvious green or red fluorescence signal in both the anterior ocular segment (cornea) and posterior ocular segment (retina), suggesting that the lipophilic small molecule and the hydrophilic small molecule are difficult to be absorbed in the eyes after being dropped into the eyes. For both 89WP-FAM and Cy5-89WP, there was no obvious corresponding fluorescence signal in the cornea area, while there was obvious green or red fluorescence signal distributions in the retinal part of the fundus, suggesting that the conjugates can be effectively absorbed and reached the retinal tissue of the fundus after being dropped into the eyes.

### 12. In Vivo Safety Evaluation of the Conjugates

### 12.1 Construction of Orthotopic Tumor-Bearing Mouse Model

Before the experiments, the healthy male Balb/c-nude mice (4 weeks old) were subjected to ophthalmic examination to ensure the normal eyes. Amobarbital sodium (40 mg/kg) was injected intraperitoneally to the mice for general anesthesia, the commercially available 0.4% oxybuprocaine hydrochloride eye drops were administrated to the surface of the eyes for local anesthesia of the eye surface, and the commercially available 0.5% tropicamide eye drops were administrated for pupil dilation. The Fluc/GFP-Rb-1 cells (resuspended in 10 mM PBS at a density of 1×10⁴ cells/μL) were injected into the vitreous body of the mice via a micro-syringe (33 G, Hamilton), 2 μL cell suspension for each eye.

As shown in FIG. 18, the male Balb/c nude mice strain were selected, and the tumor cells (2 μL for each eye, containing 2×10⁴ cells) were injected into the vitreous body near retina via a micro-syringe (33G, Hamilton). For the tumor-bearing mice, administration was performed by ocular surface dropping. Compared with the eyeballs of the normal mice, there is a distribution of white diffuse tumor cells in the eyes of the tumor-bearing mice.

### 12.2 Dosage Regimen and Evaluation

Dosage regimen: the mice with similar bioluminescent signal intensity were randomly divided into five groups, including: 1) normal saline eye dropping group, 2) melphalan solution eye dropping group, 3.0 mg/mL, 3) low concentration 89WP-Mel solution (containing 0.3 mg/mL melphalan) eye dropping group, 4) high concentration 89WP-Mel solution (containing 3.0 mg/mL melphalan) eye dropping group; the medium for all the eye drops is normal saline, and 10 μl of the eye drops was dropped into the conjunctival sac of mice, once a day; 5) melphalan solution intravitreal injection group, 8.0 mg/mL, 2 μL intravitreal injection was taken as a positive control.

Detection scheme: At the end of 1 month administration, the mice were euthanized by an overdose of anesthesia (anaobarbital sodium, 150 mg/kg). The cardiac perfusion was performed sequentially with normal saline and 4% paraformaldehyde. The eyeballs, hearts, liver, spleen, lung, kidney, brain and other organs were harvested and dehydrated overnight in 15% and 30% sucrose solutions gradient. The HE stained sections and immunohistochemical sections were made and observed.

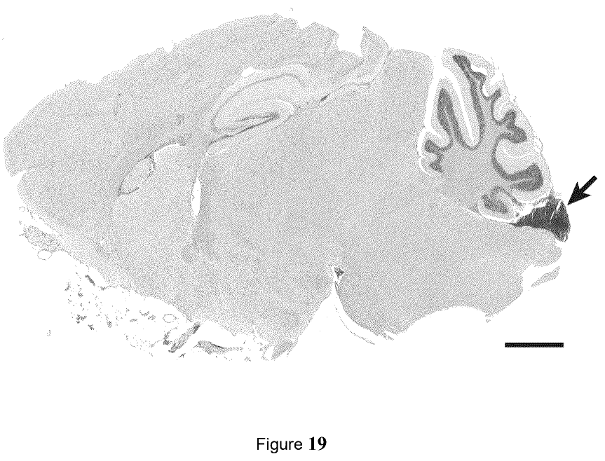

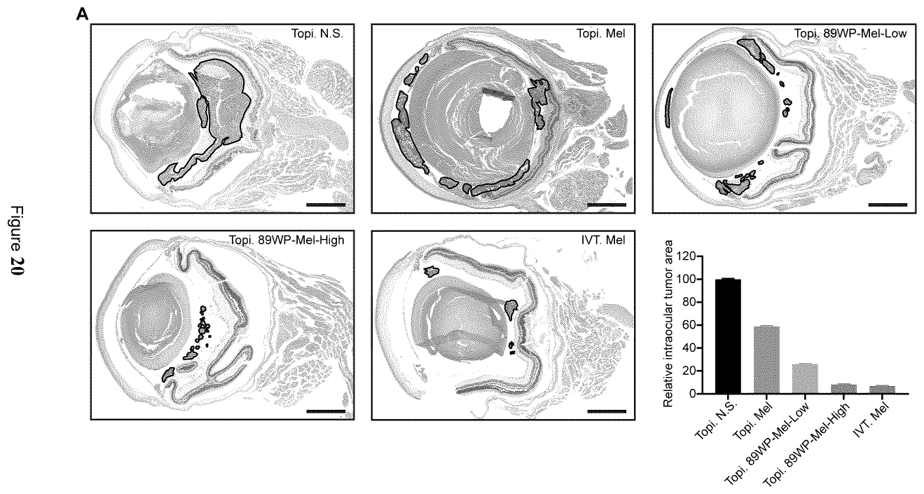

According to the results of brain immunohistochemical sections (FIG. 19), there were GFP-labeled tumor cells in the spinal trigeminal nucleus area of the mice brain, confirming that the intraocular tumors have metastasized to the brain, probably via optic nerve pathway. In the later stage, further malignant proliferation of tumor cells cause death of the mice. Due to the small sample size (n=5), the significance analysis was not performed on the survival of each group. However, based on the current results, the survival period of the mice in low and high concentration eye dropping groups was prolonged compared with the melphalan solution eye dropping and injection groups.

The HE-stained mice eyeballs sections (FIG. 20) showed that, for all the administration groups, the corneal structures of the mice were intact, without epithelial edema or fiber breakage, and the tissue structure of the posterior ocular segment was intact, suggesting that the drug had no obvious toxicity on the normal tissues of the eyeballs of the mice under the given dosage regimen. Comparing the size of the “vitreous seeds” of intraocular tumors among the groups, it can be seen that: the normal saline group≈melphalan solution eye dropping group>>low concentration conjugate solution eye dropping group>high concentration conjugate solution eye dropping group≈melphalan solution injection group.

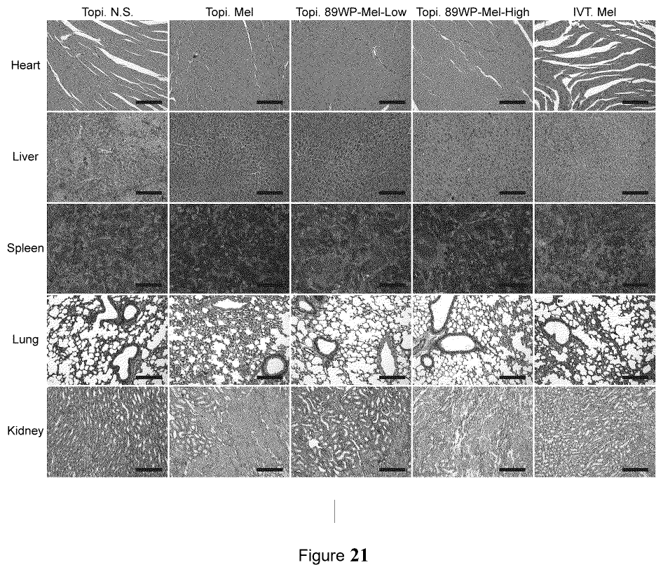

The HE-stained sections of the various organs of the mice were made, and the results are shown in FIG. 21. The structures of each organ are intact, and the nuclear and cytoplasmic staining is clear, without tissue/fiber breakage, tissue vacuole formation and tissue edema, suggesting that the application of 89WP-Mel by dropping into the eyes of mice is safe under the experimental conditions.

### 13. Intraocular Pharmacodynamic Evaluation of the Conjugates

Based on Experiment 12, the pharmacodynamic experimental results (including the anti-tumor effect and the anti-intraocular tumors brain metastasis effect) of 89WP-Mel were verified by the optimizing the experimental conditions, including the enlargement of sample size and optimization of the dosage, etc.

Dosage regimen: the mice with similar bioluminescent signal intensity were randomly divided into 4 groups, including: 1) normal saline eye dropping group, 2) melphalan solution eye dropping group, 3.0 mg/mL, 3) 89WP-Mel solution (containing 3.0 mg/mL melphalan) eye dropping group; the medium for all the eye drops is normal saline, and 10 μl of the eye drops was dropped into the conjunctival sac of the mice, once a day; 4) melphalan solution intravitreal injection group, 0.5 mg/mL, once per two weeks, 2 μL for each injection.

Detection scheme: The bioluminescence in the eyes of the tumor-bearing mice was detected by in vivo imaging technology. Specifically, D-luciferin (150 mg/kg) was injected intraperitoneally into each mouse, followed by general anesthesia with isoflurane. Twenty minutes after anesthesia, ocular bioluminescence signals were collected, with 30 seconds exposure time. On the day of administration (D0) and at the corresponding time points after administration, the bioluminescence signals of the mice eyeballs in each group were collected. At the end of the last test, the mice were euthanized by an overdose of anesthesia (amobarbital sodium, 150 mg/kg). The cardiac perfusion was performed sequentially with normal saline and 4% paraformaldehyde. The whole brain tissue was harvested. The immunohistochemical sections were made and observed.

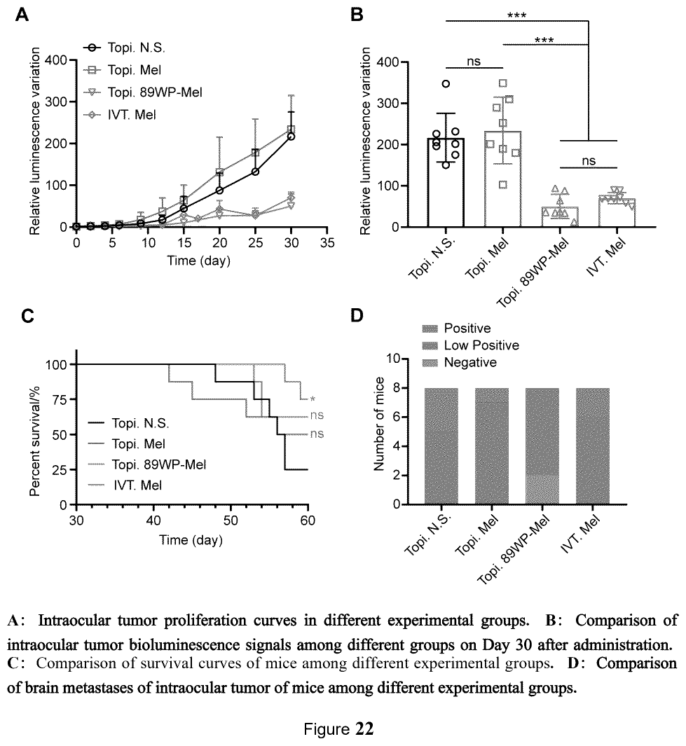

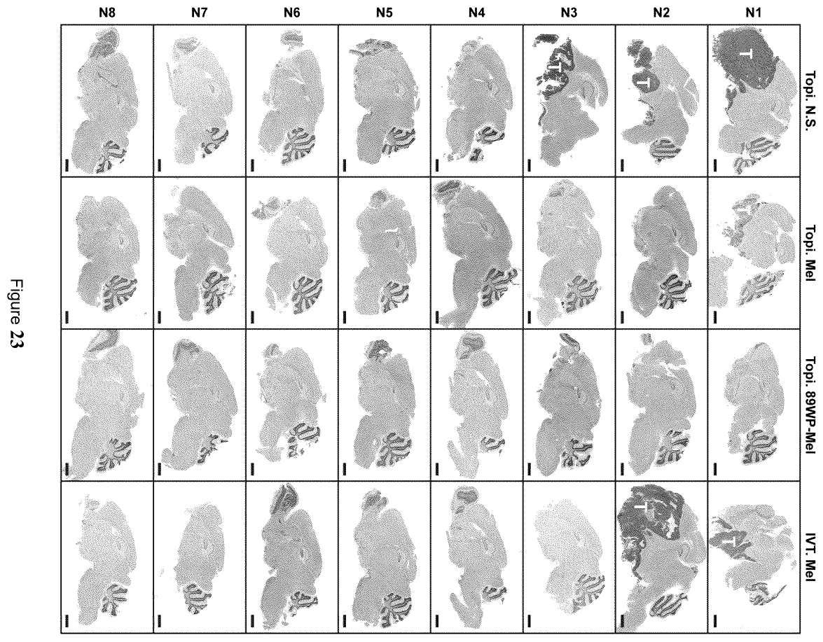

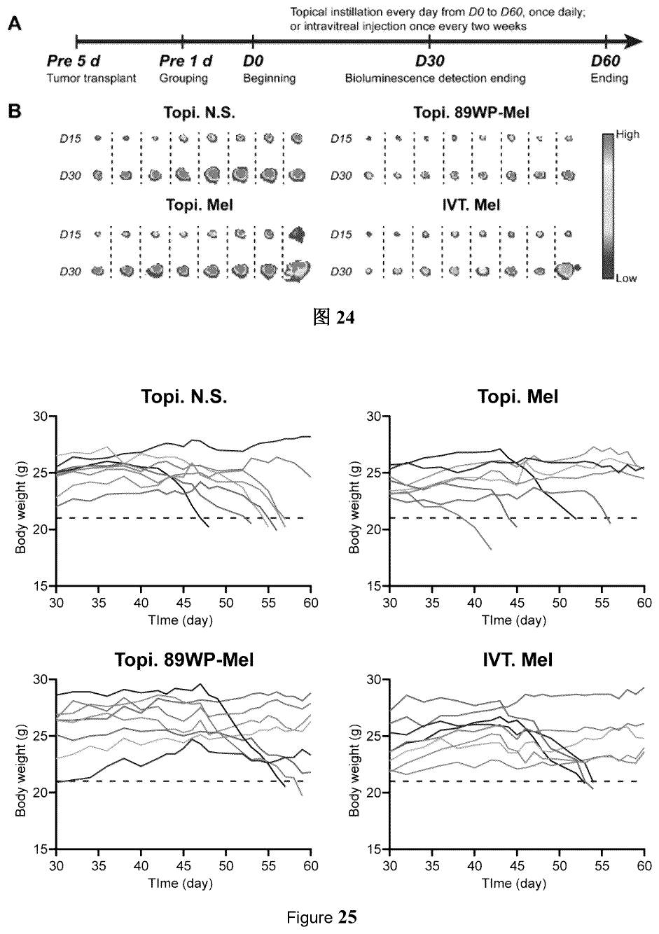

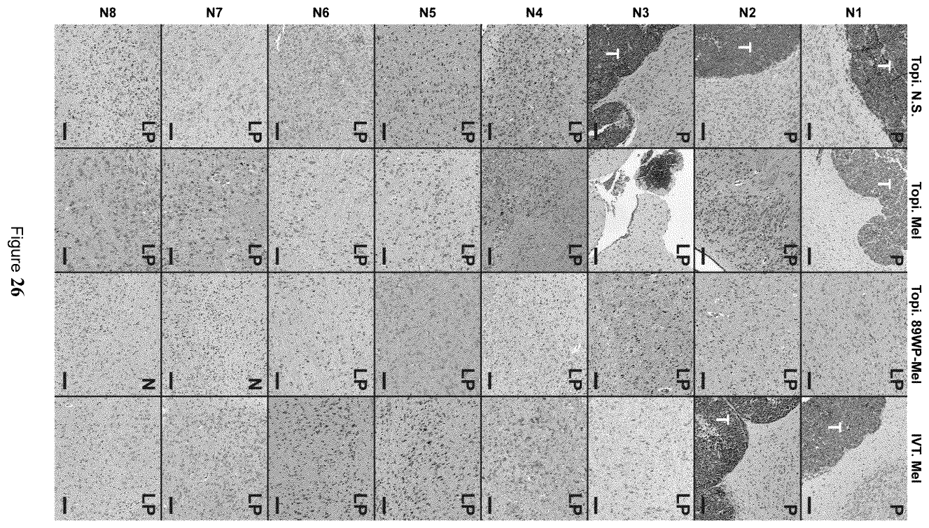

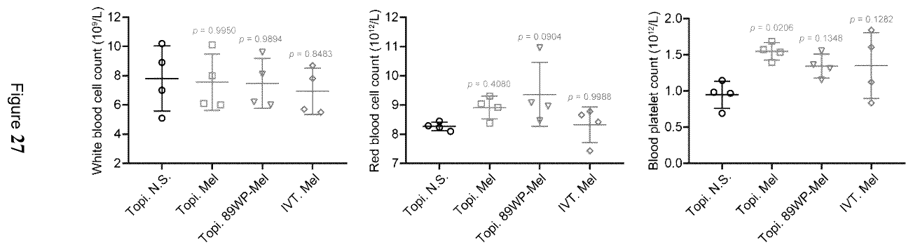

As shown in FIG. 22A, by taking the normal saline eye dropping group as the blank control, there was no significant difference in the tumor growth curve between the melphalan solution eye dropping group and the blank control. The conjugate 89WP-Mel solution eye dropping group shows an obvious effect of tumor inhibition; there was no significant different in the tumor growth within 30 days after administration between the conjugate 89WP-Mel solution eye dropping group and the melphalan solution intravitreal injection group (FIG. 22B), while the survival period of the mice in the 89WP-Mel group was significantly longer than that of the normal saline group (p<0.05). However, there was no significant difference in the survival period between the intravitreal injection group and the normal saline group (FIG. 22C). The brain metastasis of intraocular tumors in the mice of each group were compared. The results based on the section analysis (FIG. 22D, FIG. 23) show that low metastases occurred in ¾ of the mice in the 89WP-Mel eye dropping group, and brain metastases were not occurred in ¼ of them. However, there were obvious brain metastases and even large-scale infiltration growth of tumor tissue in the brains of the mice in the other groups. Three mice in the normal saline eye dropping group, one mouse in melphalan solution eye dropping group and two mice in melphalan solution intravitreal injection group had obvious brain tumor areas.

It can be seen from FIG. 22 that the in vivo efficacy in the covalent conjugate 89WP-Mel solution eye dropping group is substantially comparable to that of the conventional melphalan intravitreal injection solution. Moreover, unexpectedly, the covalent conjugate 89WP-Mel solution eye dropping group showed significant reduction in the proportion of brain metastases of intraocular tumors, and the effect thereof even exceeds the effect of conventional melphalan intravitreal injection solution; the negative ratio of brain metastases occurrence is significantly higher than the latter.

### 14. Polypeptide-Melphalan Eye Drops for the Treatment of Intraocular Retinoblastoma

Construction of retinoblastoma mouse model: the male Balb/c nude mice (18-20 g) were subjected to general anesthesia by intraperitoneal injection of amobarbital sodium (30 mg/kg). The 0.4% oxybuprocaine hydrochloride was administrated for local anesthesia of the eye surface. After the anesthesia, the 0.5% tropicamide was administrated for pupil dilation. The retinoblastoma (Fluc/GFP-Rb-1) cells (resuspended in 2 μL 10 mM PBS, 2×10⁴ cells for each mouse) were slowly injected into the vitreous body near retina via a micro-syringe (33 G, Hamilton). After the injection, 0.25% chloramphenicol was dropped to the ocular surface to prevent ocular inflammation. Then, it is determined whether the model was successfully established according to the bioluminescent signal intensity in the eyes of the mice. The successfully established model mice were randomly divided into 4 groups, with 8 mice in each group.

During the 60-day pharmacodynamic evaluation experiment, the dosage regimens for the different mice groups are as follows: Group 1: eye dropping with normal saline once a day, 10 μL for each dropping; Group 2: eye dropping with 3.0 mg/mL melphalan solution once a day, 10 μL for each dropping; Group 3: eye dropping with 89WP-Mel solution (containing 3.0 mg/mL melphalan) once a day, 10 μL for each dropping; Group 4: intravitreal injection with 0.5 mg/mL melphalan solution once every two weeks, 2 μL for each injection.

The method of efficacy evaluation is as follows: on Day 0, 2, 4, 6, 9, 12, 15, 17, 20, 25 and 30 after the administration, the mice in each group were injected intraperitoneally with 150 mg/kg D-Luciferin, followed by anesthesia with isoflurane for 15 min. The bioluminescent signal intensity in the eyes of the mice was detected by IVIS Spectrum system (PerkinElmer, USA). The mice were euthanized by CO₂ asphyxiation when the mice were in a dying state (the body weight drops rapidly below 21 g; if the mice die, the immunohistochemical section detection cannot be performed) or at the end of the experiment (Day 60). The whole brain tissue was harvested and fixed in 4% paraformaldehyde, then dehydrated in 30% sucrose solution. The immunohistochemical sections were made. The detection index is green fluorescent protein in the metastatic tumor cells.

The anti-intraocular tumor effect of 89WP-Mel eye drops was evaluated. In the first month of administration, the anti-tumor effect of 89WP-Mel eye drops was comparable to that of the melphalan intravitreal injection group (FIG. 24B), while the melphalan eye drops have no antitumor effect.

The brain metastasis of intraocular tumors was evaluated according to the results of the mice brain immunohistochemical sections. Except for the 89WP-Mel eye dropping group, there were obvious tumor areas in the brain sections of some mice in the other groups: 3 mice in the normal saline eye dropping group, 1 mouse in the melphalan solution eye dropping group, and 2 mice in the melphalan intravitreal injection group (FIG. 23). In the 89WP-Mel eye dropping group, there were two mice that completely have no metastatic tumor cell distribution in the brain sections, suggesting that the brain metastasis tendency of intraocular tumors was completely prevented by 89WP-Mel.

Safety evaluation of 89WP-Mel eye drops: it is likely that the eye drops may be absorbed systemically, while the chemotherapeutic drug melphalan may cause systemic toxicity. The representative side effects include bone marrow suppression, etc. Thus, hematology evaluation was performed in the administration groups to determine the safety of the eye drops. Healthy male ICR mice (18-20 g) were subjected to the administration according to the regimen of pharmacodynamic evaluation experiment, wherein the frequency of eye dropping the normal saline, melphalan solution, and 89WP-Mel solution was changed to 2 times a day for 2 weeks, and the melphalan intravitreal injection was performed once a week. At the end of administration, 200 μL of whole blood was taken, anticoagulated with EDTA, and subjected to a complete blood cell count.

According to the results of routine blood tests (FIG. 27), the white blood cell count, red blood cell count, and platelet count of the mice in the 89WP-Mel eye dropping group did not significantly decrease compared with the normal saline eye dropping group (which is theoretically safe for mice), suggesting that the 89WP-Mel eye drops are safe for mice at the given dose, and would not cause side effects such as bone marrow suppression.

1. A compound of formula (I), (II) or (III), or a pharmaceutically acceptable salt thereof, which represents a covalent conjugate of cell-penetrating peptide derivative and melphalan, 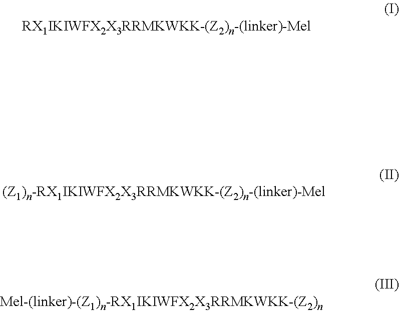
wherein, X₁, X₂ and X₃ represent hydrophobic amino acid, each of which is independently selected from alanine (A), valine (V), leucine (L), isoleucine (I), proline (P), phenylalanine (F), tryptophan (W), methionine (M) and non-naturally occurring amino acids α-aminobutyric acid, α-aminopentanoic acid, α-aminohexanoic acid and α-aminoheptanoic acid;
Z₁ and Z₂ represent naturally or non-naturally occurring amino acids, which is independently selected from 1, 2, 3, 4 or 5 of glycine (G), alanine (A), lysine (K), arginine (R), serine (S), histidine (H), aspartic acid (D), glutamic acid (E), threonine (T), proline (P), cysteine (C), tyrosine (Y), valine (V), methionine (M), isoleucine (I), leucine (L), phenylalanine (F), tryptophan (W), Glutamine (Q), Asparagin (N), and non-naturally occurring hydroxyproline, α-aminobutyric acid, α-aminopentanoic acid, α-aminohexanoic acid and α-aminoheptanoic acid, and the numbers of Z₁ and Z₂ are independent of each other;
n is an integer ranging from 0 to 10, preferably from 0 to 5, for example 0, 1, 2, 3, 4 or 5;
Mel is the melphalan moiety, and
linker represents a covalent bond or a linking group formed between melphalan and the adjacent amino acid, such as amide bond, ester bond, ether bond, disulfide bond, hydrazone, urea, oxime, guanidine, amidine, acetal, imine, alkylene or other linking forms; for example, linker can be selected from the following forms: —O—, —S—, —S—S—, —CH═N—O—, —C₁₋₆ alkylene-, —NH—, —N(R₁)—, —CO—NH—, —CO—N(R₁)—, —C₁₋₆ alkylene-(CO—NH)—, —C₁₋₆ alkylene-(CO—N(R₁))—, —C₁₋₆ alkylene-N(R₁)—, —NH—CO—, —N(R₁)—CO—, —C₁₋₆ alkylene-NH—CO—, —C₁₋₆ alkylene-N(R₁)—CO—, —NH—CO—NH—, —N(R₁)—CO—NH, —NH—CO—N(R₁)—, —N(R₁)—CO—N(R₁), —C(═O)O—, —C₁₋₆ alkylene-C(═O)O—, —C(═O)O—C₁₋₆ alkylene-, —S(—O)NH—, —S(═O)N(R₁)—, —NH—S(═O)—, —N(R₁)S(═O)—, —C₁₋₆ alkylene-S(═O)NH—, —C₁₋₆ alkylene-S(═O)N(R₁)—, —C₁₋₆ alkylene-NH—S(═O)—, —C₁₋₆ alkylene-N(R₁)S(═O)—, —S(═O)₂—NH—, —S(═O)₂—N(R₁)—, —NH—S(═O)₂—, —N(R₁)S(═O)₂—, —C₁₋₆ alkylene-S(═O)₂—NH—, —C₁₋₆ alkylene-S(═O)₂—N(R₁)—, —C₁₋₆ alkylene-NH—S(═O)₂—, —C₁₋₆ alkylene-N(R₁)S(═O)₂—, or C₁₋₆ alkylene substituted with one or more R₁; wherein R₁ is selected from C₁₋₆ alkyl, C₃₋₈ cycloalkyl, C₆₋₁₀ aryl, 5- to 10-membered heteroaryl or 3- to 12-membered heterocycloalkyl, each of which is optionally substituted with one or more groups independently selected from halogen, amino, —NH(C₁₋₆ alkyl), —N(C₁₋₆ alkyl)₂, hydroxyl, C₁₋₆ alkyl, C₂₋₆ alkenyl, C₂₋₆ alkynyl, C₁₋₆ alkoxy, halogen-substituted C₁₋₆ alkyl, halogen-substituted C₁₋₆ alkoxy, halogen-substituted C₂₋₆ alkenyl or halogen-substituted C₂₋₆ alkynyl.

2. The compound according to claim 1, which is a compound of formula (IV), wherein an amide bond connection is directly formed by the amino group of melphalan and the carboxyl group terminal of the cell-penetrating peptide derivative: 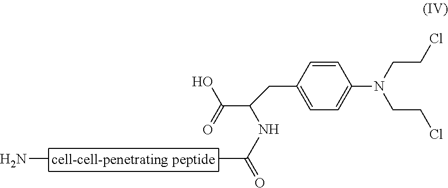
wherein the “cell-penetrating peptide” moiety is as defined for the compound of formula (I), (II) or (III) in claim 1.

3. The compound according to claim 2, which is a compound of formula (V), or a pharmaceutically acceptable salt thereof, 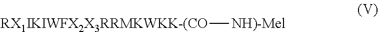
wherein, Mel is the melphalan moiety shown in formula (IV), and the free amino group of Mel forms an amide bond —(CO—NH)— with the carboxyl group of the C-terminal Lys of the cell-penetrating peptide,
X₁, X₂ and X₃ represent hydrophobic amino acids, each of which is independently selected from alanine (A), valine (V), leucine (L), isoleucine (I), proline (P), phenylalanine (F), tryptophan (W), methionine (M) and non-naturally occurring amino acids α-aminobutyric acid, α-aminopentanoic acid, α-aminohexanoic acid and α-aminoheptanoic acid.

4. The compound according to claim 3, wherein any one of X₁, X₂ and X₃ of the compound of formula (V) is tryptophan.

5. The compound according to claim 3, wherein any two of X₁, X₂ and X₃ of the compound of formula (V) are tryptophan.

6. The compound according to claim 3, wherein all of X₁, X₂ and X₃ of the compound of formula (V) are tryptophan.

7. The compound according to claim 3, wherein the compound of formula (V) is selected from the following compounds or a pharmaceutically acceptable salt thereof:

| Number | Sequence |
|---|---|
| 2WP-Mel | RWIKIWFQNRRMKWKK-(CO-NH)-Mel |
| 8WP-Mel | RQIKIWFWNRRMKWKK-(CO-NH)-Mel |
| 9WP-Mel | RQIKIWFQWRRMKWKK-(CO-NH)-Mel |
| 28WP-Mel | RWIKIWFWNRRMKWKK-(CO-NH)-Mel |
| 29WP-Mel | RWIKIWFQWRRMKWKK-(CO-NH)-Mel |
| 89WP-Mel | RQIKIWFWWRRMKWKK-(CO-NH)-Mel |
| 289WP-Mel | RWIKIWFWWRRMKWKK-(CO-NH)-Mel |

8. The compound according to claim 3, wherein the compound of formula (II) is selected from the following compounds or a pharmaceutically acceptable salt thereof:

| Number | sequence |
|---|---|
| 89WP-Mel | RQIKIWFWWRRMKWKK-(CO-NH)-Mel |
| 289WP-Mel | RWIKIWFWWRRMKWKK-(CO-NH)-Mel |

9. A pharmaceutical composition comprising a compound according to claim 1 or a pharmaceutically acceptable salt thereof, and pharmaceutically acceptable excipients or carriers.

10. The pharmaceutical composition according to claim 9, which is in the form of a liquid pharmaceutical composition.

11. The pharmaceutical composition according to claim 10, which is in the form of injection solution or eye drops.

12. (canceled)

13. (canceled)

14. A method for preventing or treating eye diseases, comprising administering a compound of formula (II) or a pharmaceutically acceptable salt thereof according to claim 1 to an individual in need thereof.

15. The method according to claim 14, wherein the eye diseases are selected from: tumors of eyelids, conjunctiva, various layer tissues of eyeball (cornea, sclera, uvea and retina) and appendages of the eyes (lacrimal apparatus, orbital and periorbital structures), including malignant basal cell carcinoma, meibomian gland carcinoma, squamous epithelial carcinoma, melanoma, retinoblastoma, choroidal melanoma, rhabdomyosarcoma, lacrimal gland adenocarcinoma, benign choroidal hemangioma, optic nerve glioma, neurofibroma, keratosis, nevus, dermoid tumor, cavernous hemangioma, dermoid cyst, lacrimal gland mixed tumor, and intraocular metastatic carcinoma, particularly retinoblastoma and choroidal melanoma, and also uveitis.

16. The method according to claim 14, wherein the eye disease is retinoblastoma.
